(19) United States
(12) Patent Application Publication
(10) Pub. No.: US 2023/0381443 A1
(43) Pub. Date: Nov. 30, 2023

(54) Exhalation Filtration Device with Aerosol Separator, Collection Reservoir, and Non-Rebreather Option

(71) Applicant: Groman Inc., Margate, FL (US)

(72) Inventor: Boaz Barry Groman, Boca Raton, FL (US)

(21) Appl. No.: 18/225,175

(22) Filed: Jul. 24, 2023

Related U.S. Application Data
(63) Continuation-in-part of application No. 17/734,133, filed on May 2, 2022.
(60) Provisional application No. 63/182,937, filed on May 2, 2021.

(51) Int. Cl. A61M 16/10; B01D 46/00; B01D 46/10; B01D 46/62; A41D 13/11; A61M 16/08; A61M 16/14

(52) U.S. Cl. CPC A61M 16/105; B01D 46/0005; B01D 46/10; B01D 46/62; A41D 13/11; A61M 16/0816; A61M 16/14; B01D 2265/04; B01D 2275/403; B01D 2279/40; A61M 2205/7509

(57) ABSTRACT

A filtration device (FD) having a front plastic sheet (PS), a sponge element (SE), and a filter element (FE) may be secured to a mask (M) with a “stickie” interface (AL, FS; FIGS. 5 and 6), or with a rigid mechanical interface (FIGS. 7 and 8) which may include a rigid support (RS) (FIG. 9). The rigid support (RS) may have a recess for receiving the sponge element (SE) and avoiding it being compressed when the filter element (FE) is disposed between to the outlet side of the rigid support (RS) and the filter element (FE). The sponge element (SE) may be provided with a cutout (CO; FIG. 10) for accommodating the flap of a non-rebreather mask (M). A protective cover (PC; FIG. 11) having perforations (PF) and a second sponge element (SE2) may be provided on the outlet side of the filter element (FE). Methods of use.

### CROSS-REFERENCE TO RELATED APPLICATIONS

This is a continuation-in-part of U.S. Ser. No. 17/734,133 filed May 2, 2022, which is a nonprovisional filing of U.S. 63/182,937 filed May 2, 2021, incorporated in its entirety by reference herein.

### FIELD OF THE INVENTION

This invention relates to medical devices and, more particularly, to filtration devices (FD) which can be used in conjunction with devices such as a nebulizer or a respiratory face mask, to capture aerosols and the like which may be expelled by a user. Said filtration devices may be referred to as “exhalation” filtration devices.

### BACKGROUND

In the 2020 Coronavirus pandemic, treatment options for patients with shortness of breath were limited due to the fear that first line and hospital personnel would be exposed to aerosolized viral particles expelled by patients into the environment. Prior to the pandemic, aerosolized medication inhaled by healthcare providers has also been an issue which this invention also addresses.

A nebulizer is a piece of medical equipment that a person with asthma or another respiratory condition can use to administer medication directly and quickly to the lungs. A nebulizer turns liquid medicine into a very fine mist that a person can inhale through a face mask or mouthpiece.

Generally, there are three main types of nebulizers:
Jet. This uses compressed gas to make an aerosol (tiny particles of medication in the air).
Ultrasonic. This makes an aerosol through high-frequency vibrations. The particles are larger than with a jet nebulizer.
Mesh. Liquid passes through a very fine mesh to form the aerosol. This kind of nebulizer puts out the smallest particles. It's also the most expensive.

Nebulizers are especially good for infants' or small children's asthma medications. They're are also helpful when you have trouble using an asthma inhaler or need a large dose of an inhaled medication.

Nebulizers are quite commonplace in hospitals and clinics these days and you'll even find quite a number of households that have one too.

Quite simply, a Nebulizer is not an oxygen delivery device, nor is it a humidifier. A Nebulizer is a drug delivery device that can dispense medication directly into the lungs in the form of an inhalable mist.

Nebulizers are used to treat various lung diseases such as: asthma, cystic fibrosis, chronic obstructive pulmonary disease (COPD), and other severe forms of lung infections and diseases.

The Nebulizer machine uses a mixture of processes involving oxygen, compressed air, and even ultrasonic power to atomize and vaporize liquid medication into small aerosol droplets, or a mist, that can be inhaled directly into the lungs.

There are three main types of electrical Nebulizers which can currently be found.
The Ultrasonic Wave Nebulizer is one of the first types of electrical Nebulizer that were designed and available on the market from 1964. An electronic oscillator creates a high frequency ultrasonic wave which causes mechanical vibrations in a piezoelectric element that breaks the liquid medication up into a fine mist. Because no air compression is used during this process, this type of Nebulizer is one of the quietest machines available.
Jet Nebulizers are one of the most commonly used machines available today. A Jet Nebulizer is also known as an atomizer because it uses compressed air to run through liquid medication at high speed, which allows it to be turned into an aerosol. Jet Nebulizers are commonly used by patients who are unable to use MDIs (metered-dose inhalers—the inhalers you usually see asthma sufferers using), or patients who require daily treatments—for whom MDIs can become very expensive. Although its big drawbacks are size, weight and noise, the Jet Nebulizer's big advantage is its low operating costs. And manufacturers are constantly improving on design and reducing overall weight and size, making the machine more portable.
The Vibrating Mesh Technology Nebulizer is one of the latest innovations in the market and it uses a laser-drilled mesh membrane which vibrates to refine the droplet size and force the liquid through, thereby creating a very fine mist. This technology allows for faster processing and thus decreases treatment times significantly. Some of the advantages of the VMT Nebulizer is that is decreases the amount of liquid waste as well as the undesired heating of the medical liquid. It is however far more expensive than any of the other types of Nebulizers which is one of its greatest drawbacks.

See https://omnisurge.co.za/what-are-nebulizers-and-how-do-they-work/In

some examples of the invention described below, a Jet Nebulizer may be shown as an exemplary nebulizer, working in conjunction with the Filtration Device disclosed herein. The invention may be suitable for use with other types of nebulizers. An example of a nebulizer is the Hudson RCI 1724 Up-Draft Nebulizer.

The nebulizer has the following major components:
a mouthpiece (at one end) through which a patient may inhale mediated air and exhale;
a T-shaped coupling component (middle);
a reservoir containing medication disposed below the T-shaped component;
a tube (at the other end; aka “exhaust tube”) for carrying air exhaled by the patient

Generally, compressed air may be delivered to the reservoir, via the hose (shown at the bottom of the reservoir), and aerosolized medication is provided to the coupling component. A valve or valves (not shown) may be incorporated into the T-shaped coupling component to (i) direct the aerosolized medication through the mouthpiece, when the patient inhales, and to (ii) direct the patient's exhaled air out the exhaust pipe to the environment.

nebulizer with mouthpiece, along with a bacterial/viral filter, and illustrates that a patient may inhale clean aerosolized air from the nebulizer, and may exhale contaminated air to the environment, preferably through the filter.

The B/V filter may be 4444/01BAUA Bacterial/Viral filter for nebulizer therapy machine “Bacterial/Viral filter for nebulizer therapy machine clinic clean pouch packed” by GVS, or the like. see http://www.gvs.com/product-family/160/963/4444

Nebulized therapy is often called a breathing treatment. You can use nebulizers with a variety of medications, both for controlling asthma symptoms and for relief right away. These include:
Corticosteroids to fight inflammation (such as budesonide, flunisolide, fluticasone, and triamcinolone)
Bronchodilators to open your airways (such as albuterol, formoterol, levalbuterol, and salmeterol)

Providing filtration of air exhaled by the patient during these treatments is very important, as evidenced by the following: “RESPIRATORY CARE OF THE NONINTUBATED PATIENT” See https://www.uptodate.com/contents/coronavirus-disease-2019-covid-19-critical-care-is sues
Nebulized medications (spontaneously breathing patients)— Nebulizers are associated with aerosolization and potentially increase the risk of SARS-CoV-2 transmission. In patients with suspected or documented COVID-19, nebulized bronchodilator therapy should be reserved for acute bronchospasm (e.g., in the setting of asthma or chronic obstructive pulmonary disease [COPD] exacerbation). Otherwise, nebulized therapy should generally be avoided, in particular for indications without a clear evidence-base; however some uses (e.g., hypertonic saline for cystic fibrosis) may need to be individualized. Metered dose inhalers (MDIs) with spacer devices should be used instead of nebulizers for management of chronic conditions (e.g., asthma or COPD controller therapy). Patients can use their own MDIs if the hospital does not have them on formulary.
If nebulized therapy is used, patients should be in an airborne infection isolation room, and healthcare workers should use contact and airborne precautions with appropriate personal protection equipment (PPE); this includes a N95 mask with goggles and face shield or equivalent (e.g., powered air-purifying respirator [PAPR] mask]) as well as gloves and gown. All non-essential personnel should leave the room during nebulization. Some experts also suggest not re-entering the room for two to three hours following nebulizer administration.

A nebulizer, such as described above, is considered to be a “non-rebreathing” device, since air, once exhaled, is not re-inhaled by the patient.

Some Patent (and other) Publications

The following patents and publications may be relevant to the invention(s) disclosed herein.
U.S. Pat. No. 5,020,530 (1991-06-04; Miller)
U.S. Pat. No. 5,613,489 (1997-03-25; Miller et al.)
U.S. Pat. No. 6,062,217 (2000-05-16; Gray)
U.S. Pat. No. 7,418,962 (2008-09-02; Rao)
U.S. Pat. No. 7,559,323 (2009-07-14L Hacke et al.)
U.S. Pat. No. 8,413,651 (2013-04-09; Powell et al.)
US 2005/0217667 (2005-10-06; Dhuper et al.)
US 2010/0095958 (2010-04-22; King et al.)
US 2017/0021201 (2017-01-26; Baker et al.) FIG. 9a-d
US 2020/0360651 (2020-11-19; Naddaff et al.)
US 2021/0330912 (2021-10-28; Groman et al.)
CN 110603069 (2019-12-20)
CN 112451811 (2021-03-09)
CN 211155896 (2020-08-04)
CN 213045306 (Zhou Xiaomian) claim 6, FIG. 3
CN 211068814 (Li Wei) FIGS. 1, 2
CN 213884642 (Zhu Qiongfang; Hei Ziqing) FIG. 1
DE 19700838 (1998-07-16; Schwabe GmBH)
DE 102020111289 (Weisser Jorg et al.) FIG. 2b
IN 202141011733 (2021-04-09; Arokiaraj)
JP 2019511348 (2019-04-25)
KR 20100064396 (Park Ki Su) FIG. 5
WO 2019/243758 (2019-12-26; Khasawneh et al.)
WO 2021/203096 (Kristen Archbold) FIG. 8

Circulaire II High-Efficiency Aerosol Drug Delivery System, Westmed, 2020, 6pp

Reducing Aerosol-Related Risk of Transmission in the Era of COVID-19: An Interim Guidance Endorsed by the International Society of Aerosols in Medicine, Fink et al., Journal of Aerosol Medicine and Pulmonary Drug Delivery, Vol 30, No. 6, 2020, 6pp

Filter Considerations in the COVID-19 Era, Mike Pedro, Vyaire Medical, 15pp

### SUMMARY

The present invention may be particularly applicable to use with a handheld nebulizer with mouthpiece.

The present invention may be particularly applicable to use with a respiratory nebulizer aerosol therapy mask.

It is a general object of the invention to provide filtration of air exhaled by a patient and, more particularly, to a patient using a handheld nebulizer, or a patient wearing a respiratory nebulizer aerosol therapy mask. Other (different, additional) applications (uses) may be within the scope of the invention.

An embodiment of the invention may comprise a filtration device (FD) adapted to be used with a device such as a handheld nebulizer which will allow a patient to receive either oxygen at high flow or nebulizers at high flow, while filtering the environment from viral particles which may be exhaled by the patient.

According to an embodiment of the invention, a filtration device (FD) may comprise:
a flexible bag (B) having an inlet port adapted to be fitted onto an exhaust tube of a hand-held nebulizer and an outlet port;
a filter element (FE) disposed on the outlet port; and
optionally, a deflector component (D) disposed adjacent the inlet port. The filtration device (FD) would work without the deflector, but the deflector makes the device better because (i) it causes aerosol extraction and (ii) it seals the inlet port during inhalation. However, some nebulizers may not provide sufficient inhalation air flow and may operate better without the deflector sealing the inlet port; however, a deflector could be utilized to extract moisture out of the air by eliminating the inlet post sealing motion by utilizing a screen.

A bottom portion of the bag (B) may comprise a collection reservoir (R) for collecting aerosols and particles exhaled by a patient.

The inlet port and outlet port may be disposed on a same side of the bag. The inlet port may be slightly smaller in diameter than the exhalation port.

The inlet port and outlet port may be disposed on opposite sides of the bag.

According to the invention, generally, an Exhalation Filtration Device (FD) with a front plastic sheet (PS), an aerosol separator (AS) and optionally a collection reservoir (R) may be fitted (i) (FIG. 3A) by its inlet port (IP) onto a nebulizer exhaust tube (NET), or (ii) by an adhesive layer (AL) onto a respiratory mask (M) having a port (P or MP) and a flat surface (FIG. 5), or (iii) by an adhesive layer (AL) and flexible substrate (FS) onto a respiratory mask (M) having a port (P) and a non-flat (FIG. 6) surface. A deflector (D), stiffener component (SC) and rear plastic sheet (PS2) are disclosed. Methods of using the Filtration Device are disclosed.

According to an embodiment of the invention, an exhalation filtration device (FD) may comprise: a plastic sheet (PS) having a periphery and an inlet port (IP); a filter element (FE) disposed behind the plastic sheet; and an aerosol separator (AS) disposed between the plastic sheet and the filter element. The aerosol separator may cover the inlet port.

The filtration device may be adapted for mounting to a respiratory mask (M) having a port (P), and may further comprise: an adhesive layer (AL) disposed on a front surface of the plastic sheet (PS) for mounting the filtration device to the mask with the inlet port of the filtration device aligned with the port of the mask. The adhesive may be suitable for allowing a given filtration device to be removed from the mask, and installing another (“fresh”) one. The adhesive layer may form a seal between the filtration device and the mask. The adhesive layer may be disposed directly on the plastic sheet, around the inlet port. The adhesive layer may comprise a pressure-sensitive adhesive having a thickness of approximately 2 mil (0.002 in; 0.05 mm). A portion of the mask having the port and to which the filtration device is mounted may be substantially flat, and may be at least somewhat rigid. Reference may be made to FIG. 5.

The filtration device may further comprise: a flexible substrate (FS) disposed between the adhesive layer and the front surface of the plastic sheet and having an opening aligned with the inlet port of the filtration device. The flexible substrate may be secured to the front surface of the plastic sheet by welding or adhesively sticking the flexible substrate to the plastic sheet. The flexible substrate may be donut-shaped. A portion of the mask having the port and to which the filtration device is mounted may be substantially non-flat, and may be at least somewhat flexible. The flexible substrate helps maintain the filtration device mounted to said non-flat and/or flexible surface of the mask. Reference may be made to FIG. 6.

The filter element has a perimeter, and the periphery of the plastic sheet may be attached, such as by welding (heat sealing), to the perimeter of the filter element.

The aerosol separator may comprise a sponge element (SE).

A deflector (D) may be disposed between the inlet port (IP) and the aerosol separator (AS);

A reservoir (R) may be disposed at an interior portion of the filtration device.

Stiffening components (SC) may be added to the filtration device, such as on the plastic sheet, to control flexure during inhalation and exhalation.

The plastic sheet may comprise a thermoplastic material such as polyethylene, polypropylene, etc., which can easily and reliably be joined (welded together) using heat. The plastic sheet may have a diameter of approximately 3 or 4 inches (7.5 or 10 cm), and may have thickness of approximately 6 mil (0.006 inch; 0.15 mm).

The filter element may comprise a pad type filter, may have a diameter of approximately 3 or 4 inches (7.5 or 10 cm), and may have thickness of approximately 43 mil (0.043 inch; 1.1 mm).

The aerosol separator may comprise a sponge element (SE).

The filtration device may further comprise: a second plastic sheet (PS2) disposed on a back side of the filtration device, behind the filter element.

Various embodiments of filtration devices (FD) suitable (adapted) to be fitted (mounted) to a medical facemask (M) are disclosed herein. The facemask (M) may be a rebreather or non-rebreather mask.

According to some embodiments of the invention, the filtration device (FD) may comprise a plastic sheet (PS) having an inlet port (IP), a sponge element (SE) disposed behind the plastic sheet (PS), and a filter element disposed behind the sponge element, and may be mounted to a mask (M) having a port (P) with a “stickie” attachment comprising an adhesive layer (AL) on the inlet side of the filtration device (FD). See FIG. 5A,B. The adhesive layer may be supported by a flexible substrate (FS). See FIG. 6A,B. This may be considered to be a “flexible” mounting of the filtration device (FD) to the mask (M).

According to some embodiments of the invention, a “rigid” mounting of the filtration device (FD) to the mask (M) may be implemented by providing an external element (EE) associated with the filtration device (FD) and a “mating” internal element (IE) associated with the mask (M). The external element (EE) may have external threads, and the internal element (IE) may have internal threads. In this manner, the filtration device (FD) may be securely screwed onto the mask (M). See FIG. 7A,B and FIG. 8A,B.

According to some embodiments of the invention, the plastic sheet (PS) on the inlet side of the filtration device (FD) may be substituted with a rigid support (RS) which may be considered to be an “oversize” external element (EE) disposed on the inlet side of the filtration device (FD). See FIG. 9. The sponge element (SE) may be smaller (e.g., in diameter) than the rigid support (RS), leaving an outer annular portion of the rigid support to which the filter element (FE) may be affixed. See FIG. 9A. And, an inner portion of the outlet side of the rigid support (RS) may be recessed to receive the sponge element (SE), reducing or avoiding compression thereof when the filter element (FE) is assembled over the outlet side of the sponge element (SE). See FIG. 9B.

According to some embodiments of the invention, the sponge element (SE) may be provided with a cutout (CO) on its inlet side to allow the flap of a non-rebreather mask to move outward when a patient wearing the mask exhales. See FIG. 10.

According to some embodiments of the invention, a protective cover (PC) may be disposed on the outlet side of the filtration device (FD) to protect the filter element (FE) from contamination and protect the healthcare provider from touching a contaminated filter. A second sponge element (SE2) may be disposed between the filter element (FE) and the protective cover (PC). The protective cover (PC) may be a second plastic sheet (PS2) having perforations (PF) allowing air to exit the filtration device (FD). See FIG. 11.

The filtration device disclosed herein may provide some of the following advantages over the prior art techniques:
Provides means for separating some of the aerosol prior to reaching the filter thereby keeping the filter dryer which reduces air resistance through the filter (easier exhalation for patient)
Provides means for capturing and retaining the bio-fluids within the device so it does not pose a risk to healthcare workers (accumulates within the device and safely discarded)
When inhaling, the bag collapses under pressure and pushes the deflector onto the Handheld Nebulizer exhalation tube blocking air from entry thereby forming a non-rebreather valve. (assures patient breaths through nebulizer to maximize medication delivery)
Less bulky for easier storage (stores flat)
Reduced Weight so its is more comfortable for the patient to hold.
More environmentally friendly (less material usage)

Other objects, features and advantages of the invention(s) disclosed herein may become apparent in light of the following illustrations and descriptions thereof.

### BRIEF DESCRIPTION OF THE DRAWINGS

Reference will be made in detail to embodiments of the disclosure, non-limiting examples of which may be illustrated in the accompanying drawing figures (FIGS.). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a diagram (cross-section) of a Filtration Device, according to an embodiment of the invention.

FIG. 2 is a diagram (cross-section) of a Filtration Device, according to an embodiment of the invention.

FIG. 3A is a diagram (cross-sectional view) of an Exhalation Filtration Device, according to an embodiment of the invention.

FIG. 3B is diagram (front view) of the Exhalation Filtration Device shown in FIG. 3A.

FIG. 4 is a diagram (cross-sectional view) of an Exhalation Filtration Device, according to an embodiment of the invention.

FIG. 5A is a diagram (cross-sectional) view, and FIG. 5B is a diagram (plan view) of a filtration device (FD) suitable for mounting to a flat surface of a mask, according to an embodiment of the invention. See also Page 15 of the APPENDIX which shows the filter being attached to and already attached to the mask. FIGS. 5A and 5B may collectively be referred to as FIG. 5.

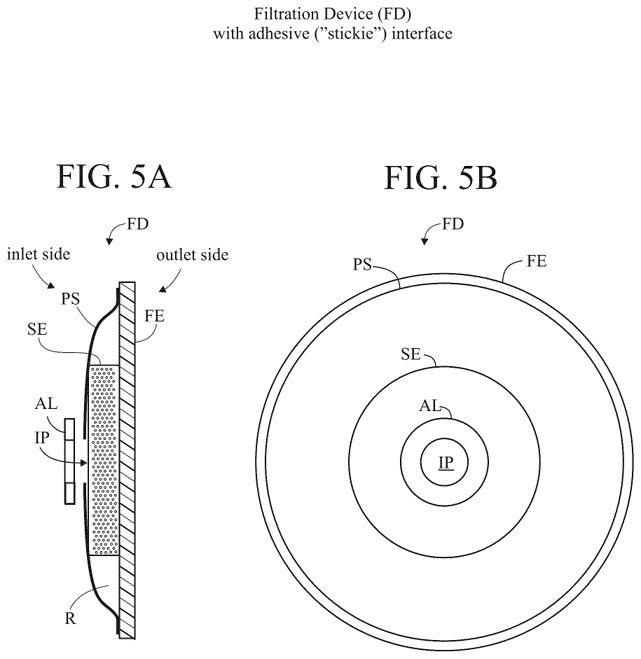

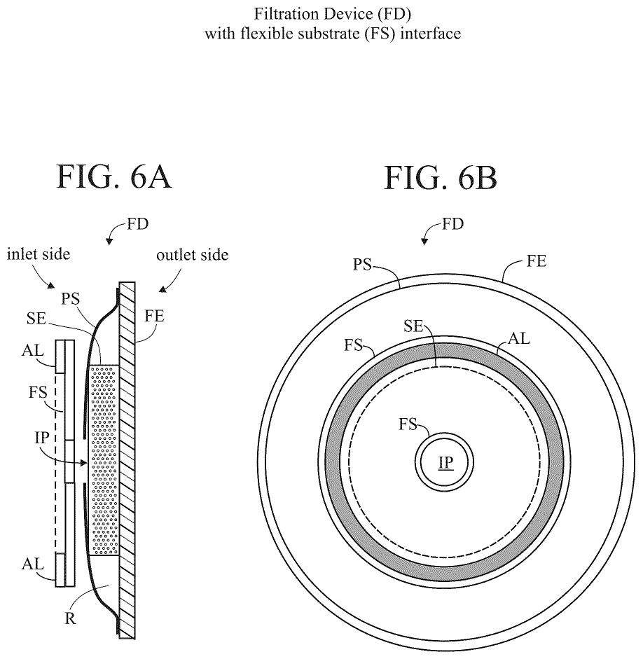

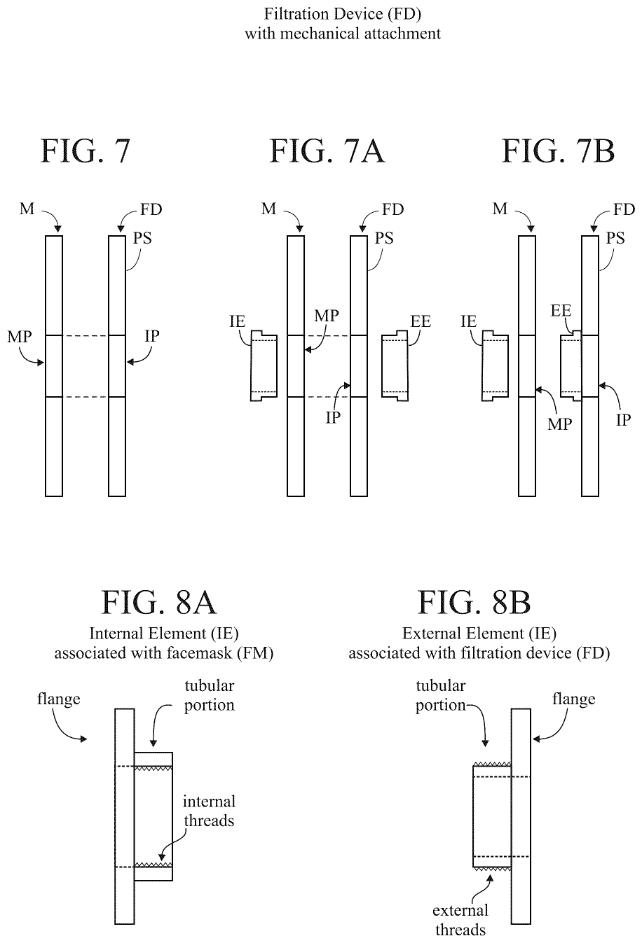

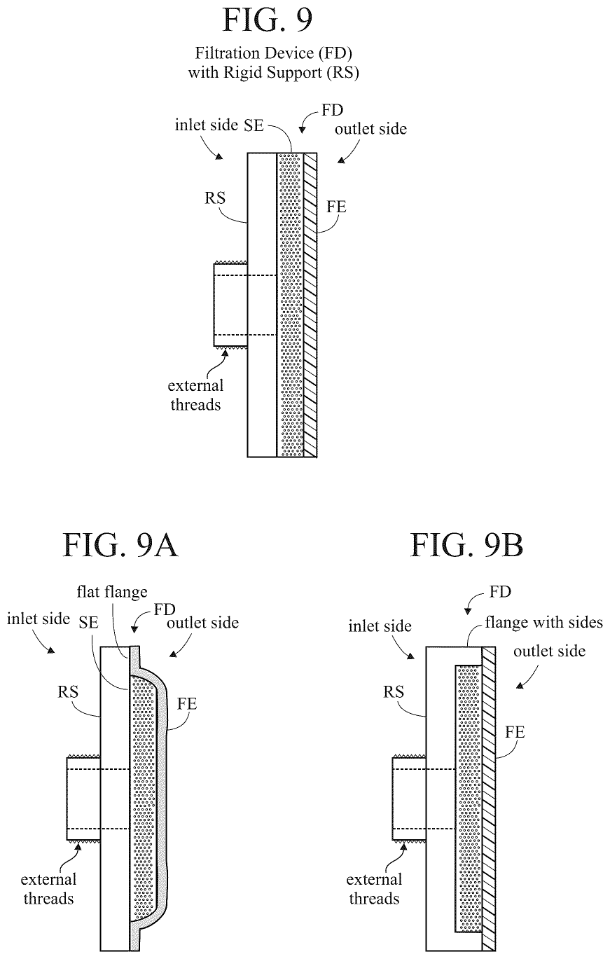

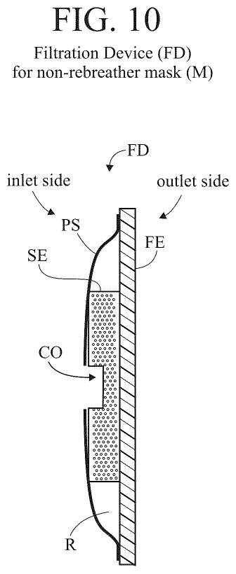

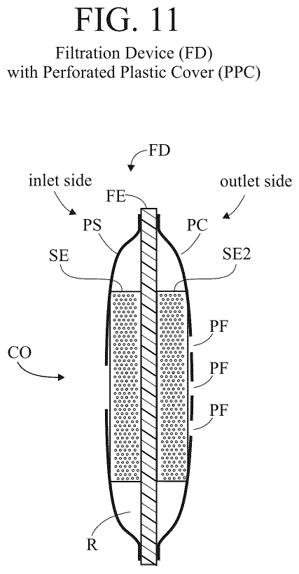

FIG. 6A is a diagram (cross-sectional view), and FIG. 6B is a diagram (plan view) of a filtration device (FD) suitable for mounting to a non-flat surface of a mask, according to an embodiment of the invention. See also Page 17 of the APPENDIX which shows the filter being attached to and already attached to the mask. FIGS. 6A and 6B may collectively be referred to as FIG. 6.
Some of the embodiments described above may include a “flexible” mounting of the filtration device (FD) to the facemask (M) with a “stickie” (adhesive) interface, such as may be shown in FIGS. 5A,B and FIGS. 6A,B. Some of the embodiments described below may include a “rigid” mounting of the filtration device (FD) to the facemask (M).

FIG. 7 is a cross-sectional view of a filtration device (FD) (only Plastic Sheet PS is shown for clarity) positioned to be mounted to a facemask (M), with the inlet port (IP) of the filtration device (FD) positioned in alignment with a port (P, or MP) of the facemask, such as described above and such as may be relevant to embodiments described below.

FIGS. 7A and 7B are cross-sectional views of a generalized embodiment or embodiments of a filtration device (FD) rigidly mounted to a facemask (M), again with the with the inlet port (IP) of the filtration device (FD) (only Plastic Sheet PS is shown for clarity) positioned in alignment with a port (P, or MP) of the facemask. Generally, in order to effectuate the rigid mounting, each of the filtration device (FD) and facemask (M) are outfitted with rigid elements that mate with one another, such as by one of the rigid elements such as an external element (EE) associated with the filtration device (FD) screwing into or onto an internal element (IE) associated with the facemask (M). FIG. 7A shows the external element (EE) flange to be on the inside of the filtration device (FD) where it abuts the internal surface of the Plastic Sheet (PS). FIG. 7B shows the external element (EE) flange to be attached to external surface of the Plastic Sheet (PS).

FIGS. 8A and 8B are cross-sectional views showing the internal (IE) and external (EE) elements with flanges, according to an embodiment of the invention. In this example the internal element (IE) associated with the facemask (M) has internal threads, and the external element (EE) associated with the filtration device (FD) has external threads. Each of the internal (IE) and external (EE) elements may comprise a tubular portion and a flange.

FIG. 9 is a cross-sectional view of an embodiment of the filtration device (FD) with a rigid support (RS), according to an embodiment of the invention.

FIG. 9A is a cross-sectional view of an embodiment of the filtration device (FD) with a rigid support (RS), according to an embodiment of the invention.

FIG. 9B is a cross-sectional view of an embodiment of the filtration device (FD) with a rigid support (RS), according to an embodiment of the invention.

FIG. 10 is a cross-sectional view of an embodiment of the filtration device (FD) having a cutout (CO) in the sponge element (SE), according to an embodiment of the invention.

FIG. 11 is a cross-sectional view of an embodiment of the filtration device (FD) having a protective cover (PC), according to an embodiment of the invention.

Some additional figures (typically photographs) are presented in the APPENDICES filed herewith, and are incorporated by reference herein, along with any text accompanying the figures.

Drawing Key and Abbreviations

The following abbreviations may be used (in the manner of reference numerals) in the text and/or drawings to identify the following items, such as the mask (M) and, more particularly, parts or components of the overall filtration device (FD), in its various embodiments.

| | | | |
|---|---|---|---|
| NET | Nebulizer Exhaust tube | M | mask |
| B/V | bacterial/viral | P | port (in the mask) |
| T | tube | PS | plastic sheet |
| B | bag | FPS | front plastic sheet |
| FD | Filtration Device | PS2 | second (rear) plastic sheet |
| F | filter | RPS | rear plastic sheet |
| FE | filter element | FM | filter media |
| D | deflector | SP | sponge |
| R | collection reservoir | SE | sponge element |
| IP | Inlet Port | IS | interface seal |
| OP | Outlet Port | FS | flexible substrate |
| EP | Exhaust Port | SS | “stickie” substrate |
| AS | Aerosol Separator | SI | “stickie” interface |
| S | sponge | AL | adhesive layer |
| D | deflector | RL | release layer |
| SC | stiffener component | PT | pull tab |
| | | PTC | pull tab cover |
| MP | mask port (see P) | IE | internal element associated |
| | | | with the facemask (M) |
| IP | inlet port of the filtration | EE | external element associated |
| | device (FD) | | with the filtration device (FD) |
| N | nut | RS | rigid support |
| FM | filter media | FD | filter (or filtration) device |
| FE | filter element | FL | flange |
| CO | cut-out in sponge element | SE2 | second sponge element |
| PC | protective cover | PF | perforations |

The filtration device (FD) may sometimes be referred to simply as “filter”, but should not be confused the filter element (F, FE) which also may sometimes be referred to simply as “filter”.

### DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

The filtration device disclosed herein is intended to filter the exhalation cycle (exhalation phase of the overall breathing cycle of inhale/exhale) in the context of:
1) providing a patient with oxygen, and/or
2) providing a patient with nebulized or aerosolized medications, with emphasis on the latter (delivering medications).

In an embodiment, the filtration device may be adapted specifically and exclusively to providing nebulized or aerosolized medications to a patient, although it may also be suitable for providing oxygen to a patient.

Nebulizer and aerosol treatments typically deliver medication to the patient. Oxygen treatments may only have gas delivery without the medication. Whatever the application, it is important to protect first responders, doctors and nurses from the patient exhalation, and also to protect the patient from any contaminants in the ambient air.

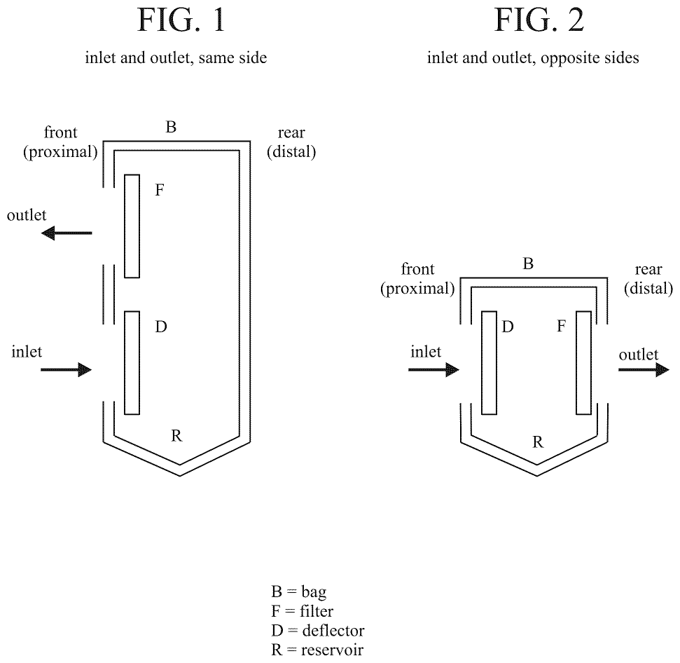

FIG. 1 shows a filtration device (FD), according to an embodiment of the invention. The device comprises:
a bag (B) having a front (proximal, to the nebulizer) wall and a rear (distal from the nebulizer) wall;
the front wall of the bag has an inlet port (opening) for coupling and being in fluid communication with the exhaust tube of the nebulizer, and receiving air exhaled by the patient (through the nebulizer);
the front wall of the bag has an outlet port (opening) for allowing air exhaled by the patient, into the bag, to escape into the ambient environment;
a deflector component (D) is disposed in the bag, at or adjacent the inlet port;
a filter component (F) is disposed in the bag, at the outlet port, and filters air exhaled by the patient into the ambient environment;
a reservoir portion (R) of the bag is provided (or reserved, or defined) at the bottom of the bag for accumulating aerosols and/or particulate matter exhaled by the patient, and deflected by the deflector component (D).

In the FIG. 1 embodiment, the inlet and outlet ports are disposed on the same side (front wall) of the bag (B).

FIG. 2 shows a filtration device (FD), according to an embodiment of the invention. The device comprises:
a bag (B) having a front (proximal, to the nebulizer) wall and a rear (distal from the nebulizer) wall;
the front wall of the bag has an inlet port (opening) for coupling and being in fluid communication with the exhaust tube of the nebulizer, and receiving air exhaled by the patient (through the nebulizer);
the rear wall of the bag has an outlet port (opening) for allowing air exhaled by the patient, into the bag, to escape into the ambient environment;
a deflector component (D) is disposed in the bag, at or adjacent the inlet port;
a filter component (F) is disposed in the bag, at the outlet port, and filters air exhaled by the patient into the ambient environment; The filter (F) can be disposed inside or outside as long as the periphery of the filter (F) is attached to the Outlet Port.
a reservoir portion (R) of the bag is provided (or reserved, or defined) at the bottom of the bag for accumulating aerosols and/or particulate matter exhaled by the patient, and deflected by the deflector component (D).

In the FIG. 2 embodiment, the inlet and outlet ports are disposed on opposite sides (front and rear walls) of the bag (B).

A filtration device (FD) conforming to the FIG. 2 embodiment (inlet and outlet ports on opposite sidewalls of the bag) may be manufactured from a single flat sheet of flexible plastic material, as follows.
An inlet port (IP) may be formed in one portion of the sheet, such as a lower portion.
An outlet port (OP) may be formed in another portion of the sheet, such as an upper portion.
The upper and lower portions may be sized and shaped similar to each other, such as square (height=base dimension), the overall sheet being rectangular (e.g., height=2×base dimension).
The filter component (F) may be disposed over the outlet port, and may be mounted securely (such as welded or glued) to the upper portion.
The sheet may then be folded, resulting in there being one common edge (bottom edge of the upper portion, upper edge of the lower portion), and the remaining three edges of the respective lower and upper portions may be sealed to one another, resulting in an air-tight bag (except, of course, for the inlet and outlet ports.)
components, such as a deflector component (D) may be located in the “nascent” proto-bag, prior to (or during) folding. The deflector component may be sized and shaped to fit within the bag, in a generally fixed position adjacent the inlet port, and the primary function of the deflector component is to separate aerosols or particles from the patient's exhaled air, allowing the aerosols of particles to drop down into a reservoir portion (R) of the bag (B).

Dimensions, Materials

The following dimensions and materials may be suitable for the Filtration Device.

For the plastic components (plastic sheets, etc.), suitable materials would be thermoplastics—Polyethylene, Polypropylene, etc., which can easily and reliably be joined (welded together) using heat.

Dimension are based on the filter size and inlet port size for creating a tight seal around the nebulizer tube and the ability for minimal bag movement during exhalation so air can flow around deflector to the filter. Approximately 4 inch (10 cm) diameter for a circular filter.

The Filtration Device (FD) disclosed herein is intended to be inexpensive, easy to use, and disposable (single use).

Appendices

Provisional application 63/182,937 was filed with two appendices:
Appendix 1 (7 pages)
Appendix 2 (14 pages), which was inclusive (cumulative) of Appendix 1

The above 2 documents are not included in this filing. Rather, 3 documents entitled Appendix-1, Appendix-2 and Appendix-3 are included in this filing. Appendix-1 was included in the filing of parent Ser. No. 17/734,133. Appendix-2 and Appendix-3 are new to this filing. These three documents (Appendices-1,-2,-3) are described hereinbelow.

Appendix-1

Included with this filing is a document (Appendix-1) entitled “Non-Rebreather Exhalation Filtration Device with Aerosol Separator and Collection Reservoir”. The Appendix-1 document is incorporated by reference herein and forms part of the Specification (description) hereof. The Appendix-1 document comprises 23 pages. Pages 1-14 of the Appendix-1 document reprises Appendix 2 of the provisional which, in turn, reprised Appendix 1 of the provisional. Pages 15-23 of the Appendix-1 document are “new” in that they disclose an “update” to the design(s) disclosed in Appendices 1 and 2 of the provisional, with some changes, additions and/or clarifications, and present some additional or alternative embodiments of the invention.

The Appendix-1 document is discussed below. The Appendix-1 document contains text. Some changes or “editorial comments” may be presented herein. The Appendix-1 document contains illustrations, which may be considered to be “Drawings” (or Figures of the Drawing), some of which correspond to drawing figures presented herein.

Discussion of the Appendix-1 document

Page 1

This page shows (i) a handheld nebulizer, and (ii) a bacterial/viral filter, of the prior art.

Left—Handheld Nebulizer with Mouthpiece and Bacterial/Viral Filter

Due to COViD these (nebulizer+filter) have been integrated to filter the patient's exhalation

Right
Inhalation Draws Clean Aerosolized Air from Nebulizer
Exhalation Direct Contaminated Air to Filter

Some problems with this prior art may be that . . .
Current inline B/V filters are bulky and heavy to be placed and the end of the nebulizer tube and take up substantial storage and shipping cost.
Current inline B/V filters get quickly saturated.
Current inline B/V filters do not have means of collecting bio-fluids
Current inline B/V filters don't have the non-rebreather option.

This page illustrates an Exhalation Filtration Device (FD) with Aerosol Separator and Collection Reservoir.

The filtration device (FD) may comprise three (3) components (elements):
a plastic sheet (PS) having an inlet port (IP);
a filter element (F, or FE) disposed behind the plastic sheet; and
a sponge (S, or SE) disposed between the plastic sheet and the filter element;
wherein the plastic sheet and filter element are joined, such as by welding, around their perimeters, with the filter element disposed therebetween.

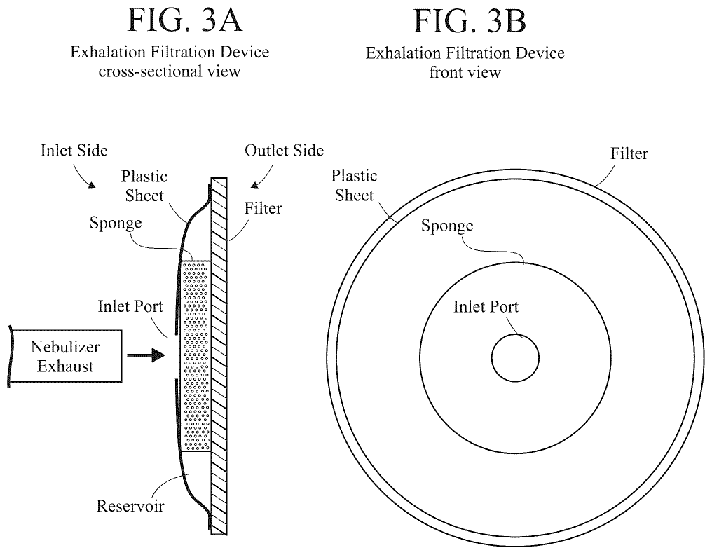

The device depicted here is also shown at FIGS. 3A,B.

The filter element (FE) may be a Blended Synthetic Fiber and Laminate Scrim of Polypropylene, and may be round, measuring 3″ in (7.5 cm) in diameter, and 43 mil (0.043 inch; 1.1 mm) in thickness.

The plastic sheet (PS) may be a sheet of a plastic material such as polyethylene, and may be round, measuring approximately 3 inches (7.5 cm) in diameter, and 6 mil (0.006 inch; 0.15 mm) in thickness. The overall diameter of the filtration device (FD) may also be approximately 3 inches (7.5 cm). All dimensions set forth herein are approximate and exemplary.

The filter (FE) may be slightly larger (in diameter) than the plastic sheet (PS), or vice-versa.

The perimeter (periphery) of the plastic sheet may be securely attached to the perimeter of the filter, such as by ultrasonic welding, impulse heating, or with an appropriate adhesive.

The sponge element (SE) may be a flat sponge, approximately 0.25″ thick. It may be round, smaller in diameter than the filter or the plastic sheet, and may be disposed between the plastic sheet and the filter prior to welding the sheet to the filter. The purpose of the sponge is to
capture/separate out aerosol or other particles
act as a spacer between the end of the nebulizer exhaust tube (NET) and the filter (F)
act as a deflector (D)/aerosol separator (AS) most of the air will go around the sponge, some may go through it air passing through the porous sponge may be dispersed on its way through the sponge
resiliently secure the Filtration Device to the nebulizer exhaust tube
act as a stiffener/support for the plastic bag so when connecting the Exhalation Tube the Inlet Port flexes in ward stretching the Inlet Port so it mounts over the Exhalation Tube. Refer to Sponge Features Graphic on Page 12

An inlet port (IP) is shown. The inlet port is essentially an opening in the plastic sheet. The opening may be slightly smaller than the nebulizer exhaust tube.

The diameter of the nebulizer exhaust tube is typically approximately 0.875 inch (2.2 cm) OD.

All dimensions set forth herein are approximate, and exemplary. By way of non-limiting example, a round filtration device may be 3 inches (7.5 cm) in diameter, +/−1 inch (2.5 cm).

Notably, the diameter of the filter is many (such as 2-6) times greater than the diameter of the nebulizer exhaust tube (NET; aka exhalation tube), or the exhalation end of a conventional B/V filter such as shown on Page 1.

The plastic sheet (PS) may be welded directly to the filter (F) with the sponge in between.

This embodiment of the filtration device (FD) works well when the filter size is larger than the side of the Handheld Nebulizer exhalation tube (NET)—in other words, a relatively large filter (FE) and a relatively small inlet port (IP). This allows the plastic sheet (PS) to wrap around the sponge (SE) without deforming the filter (FE).

Refer to Page 4 for a Non-Rebreather configuration (with additional deflector (D)).

The filtration device (FD) described above (APPENDIX; Page 2) has 3 components—filter element (F, FE), sponge (S), and (front) plastic sheet (PS). Page 3 shows an embodiment with an extra (fourth) component added. The extra component is an additional plastic sheet (PS2) on the back (rear, opposite) side of the filtration device (FD), behind the filter element (FE).

In the 3-component embodiment, the plastic sheet with the inlet port (IP) is attached to the filter element (FE). In the 4-component embodiment, there is another plastic sheet (PS2) that supports an outlet port (OP). This plastic sheet (PS2) may be attached to the filter element (FE), and both plastic sheets (PS, and PS2) may be attached (joined, such as by welding) at their peripheries.

The four illustrations across the top of Page 3 show, from left-to-right . . .
the inlet port side of the Filtration Device, showing the front plastic sheet (PS) with a small hole which is the inlet port (IP) which corresponds with the diameter (OD) of the nebulizer exhaust tube (NET)
the outlet port (OP) side of the Filtration Device (FD), prior to filter attachment
the outlet port side of the Filtration Device (FD), after filter attachment
the aerosol separator (or sponge element (SE))

The back plastic sheet (PS2) may be either in front of or behind the filter.

The sponge element (SE) serves 3 functions/purposes: (i) as an aerosol separator, (ii) as a spacer to keep the nebulizer exhalation tube (NET) spaced away/apart from the filter element (FE) to eliminate the opportunity for obstruction/restriction, and (iii) to support the front plastic sheet (PS) during insertion of the nebulizer exhalation tube (NET) into the inlet port (IP). Refer to Page 12 for a more detailed representation of the nebulizer exhalation tube (NET) being inserted into the inlet port (IP) of the front plastic sheet (PS).

From the first two illustrations, it is apparent that there are two plastic sheets (PS, PS-1, PS-2). These two sheets are both in the form of discs, with holes at their center, and the two sheets may be welded together around their perimeter (circumference) to form a bag, with the aerosol separator or sponge disposed between the two plastic sheets, in the bag.

Whereas the Page 2 embodiment had only a single plastic sheet with an inlet port, this (Page 3) embodiment has two sheets of plastic, joined around their peripheral edges, to form a bag.

The bag (B) comprises:
a front plastic sheet (PS, FPS), or a first sheet (or side), which may be in the shape of a disc, constituting the inlet side of the Filtration Device, having: an outside diameter (OD) approximately the same size (diameter) as the filter component and a hole in the center of the disc, the hole having a diameter approximately the same size as the nebulizer (or page 1 filter) exhaust tube. (Note: when using the Filtration Device of the present invention, it is not necessary to use the filter shown at page 1, and the Filtration Device would commonly be installed directly on the nebulizer exhaust tube.)
a rear plastic sheet (PS, PS2, RPS), or a second sheet (or side), which may be in the shape of a disc, constituting the inlet side of the Filtration Device, having: an outside diameter (OD) approximately the same size (diameter) as the OD of the first sheet, and hole in the center of the disc constituting the outlet port of the Filtration Device. Rather than having a single outlet port, a number of holes may be formed n the second sheet to allow filtered air to be exhausted from the Filtration Device.

The Filtration Device (FD) may further comprise:
an aerosol separator, aka filter element, disposed between the first and second sheets.

The Aerosol Separator (AS) is made of open cell sponge low flow resistance material such as used in air conditioning systems for air filtration:
Air navigates through “billions” of randomly placed open cells thereby maximizing aerosol extraction on way to the B/V filter element.
Sponge material resists deformation thereby keeping the B/V filter at a distance from the Nebulizer Exhaust Tube (NET) to avoid restriction.
Sponge material deflects at the Inlet Port when Nebulizer Exhaust Tube is inserted, allowing the Inlet Port to be pushed over the Nebulizer Exhaust Tube, thereby eliminating the extra component on previous prototypes (refer to deflector (D) in the Appendix 1 embodiment) that facilitate the pulling of the Inlet Port over the Exhaust Tube.
Collapses flat for packing especially when vacuum-packed with air removed from bag.

The three illustrations across the bottom of the page show, from left-to-right . . .
the Filtration Device (FD) mounted to a nebulizer exhaust tube (NET)
a diagram illustrating that flow may be into or out of the FD through its inlet port (IP), although flow out of the inlet port may be restricted by the aerosol separator (AS)
the assembled Filtration Device (FD), as viewed from the inlet side. Inlet Port Side is attached at the periphery to the Outlet Port (OP) Side (with the Filter Attached) and the Aerosol Separator (AS) captured between the two sides.

Page 4 This page illustrates a non-rebreather embodiment of an exhalation filtration device (FD). This embodiment is similar to the embodiment at Page 3, with the addition of a deflector element (D), which functions as a sort of check valve to allow air to be exhaled through the filtration device (FD) without allowing air to be inhaled through the filtration device (FD), hence the term “non-rebreather”.

This embodiment of the filtration device (FD) has a separate, additional non-rebreather deflector element (D, or NRD) which may act as a sort of check valve, making the filtration device (FD) a “non-rebreather” apparatus. The non-rebreather deflector (D) element facilitates the non-rebreather (i.e., one way, patient exhale only) operation of the filtration device (FD). The non-rebreather deflector (D) may deflect aerosols and particles by causing aerosols and particles impinging upon the rebreather deflector (D) during patient exhalation to be prevented from impinging on the aerosol separator (AS) or sponge element (SE) and onto the filter (F, causing the aerosols and particles to drip down (by gravity) into a reservoir (R) bottom portion of the filtration device (FD).

The previous (Page 3) embodiment has two sheets of plastic (PS, PS2), joined around their peripheral edges, to form a bag (B), and an aerosol separator (AS) disposed in the bag.

In this embodiment, a deflector (D) is disposed (located) between the inlet port (IP) in the front plastic sheet (PS) and the aerosol separator (AS) or sponge element (SP) and is also enclosed within the filtration device (FD).

When the patient inhales through the nebulizer (page 1), with the filtration device (FD) in place on the nebulizer exhaust tube (NET), the deflector (RD) is drawn towards the patient, blocking the inlet port (IP) and/or creating a seal with the nebulizer exhaust tube (NET) inserted through the inlet port (IP) so that the inlet port (IP) is blocked/sealed during inhalation.

When the patient exhales through the nebulizer, with the filtration device (FD) in place on the nebulizer exhaust tube (NET), the deflector (D) is pushed away from the patient to permit air flow through the inlet port (IP), around the deflector (D), and through the aerosol separator (AS) or sponge element (SP).

The two illustrations on the bottom of Page 4 show the position of the non-rebreather deflector (D, NRD), and subsequent blocking (during inhalation) or preventing (during exhalation) of air flow through the filtration device (FD).

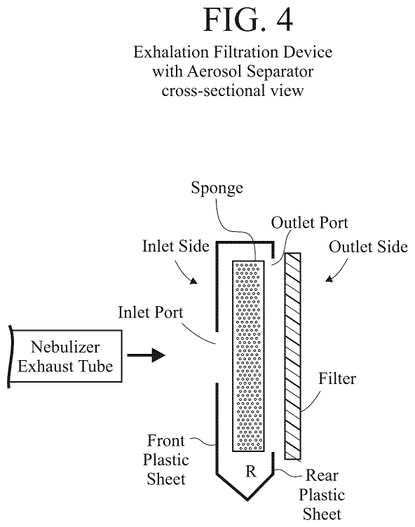

FIG. 4 shows the filtration device (FD), which comprises:
a front plastic sheet (FPS, or PS), comparable to the sole plastic sheet (PS) in the Page 3 embodiment
   the front plastic sheet is provided with an inlet port (IP), like the Page 3 embodiment
a filter (F), comparable to the filter F in the Page 3 embodiment
an aerosol separator (AS) or sponge, comparable to the AS in the Page 3 embodiment and which further comprises:
a rear plastic sheet (RPS), which was not present in the Page 3 embodiment
a non-rebreather deflector (NRD)

In FIG. 4, the filter (F) is shown “exploded” away from the outlet port (OP) on the rear plastic sheet (RPS, or PS2), for illustrative clarity.

Some differences between the Page 3 (previous) and Page 4 (this) embodiments may be noted:
In this embodiment, the filter element (F, or FE) and aerosol separator (AS) or sponge (SP) may have substantially the same cross-dimension (e.g., diameter) as one another.
the Non-Rebreather Deflector (NRD) is not present in the Page 3 embodiment

The bag, indeed the entire Filtration Device (FD) is intended to be inexpensive to manufacture, and readily disposable (e.g., “single use”).

The front and rear plastic sheets (PS, and PS2, respectively) may be joined (such as heat sealed, or welded) at their peripheral edges to form a bag (B), having an inlet port (IP) and an outlet port (OP), otherwise air-tight, and containing the filter (F) and the non-rebreather deflector (NRD).

A bottom portion of the bag is shown, serving the purpose of a reservoir (R) for collecting aerosols and/or particles separated by the aerosol separator (AS) or sponge (SP).

The reservoir (R) may be seen at Pages 5 and 6.

Page 5

This page shows an embodiment of an Exhalation Filtration Device (FD) with Aerosol Collection Reservoir (R). The illustration on the left shows the nebulizer, with a filtration device fitted to the exhaust tube (NET) of the nebulizer.

The illustration on the left of Page 5 shows the filtration device (FD) mounted on a hand-held nebulizer. The reservoir (R) is at the bottom of the bag (circled), and may be any interior portion (volume) of the bag (B) that can collect droplets. It need not be a separate component.

An exemplary (illustrative) filtration device (FD) of the present invention may comprise:
(i) a bag, adapted to fit over the exhaust tube of a nebulizer;
the bag may have two thin plastic walls, joined around their entire perimeter;
one wall of the bag may be provided with an opening (“inlet port”) for fitting over the distal end of the exhaust tube of the nebulizer. Note that the filtration device may be “substituted” for the bacterial/viral filter shown on Page 1. The Filtration Device disclosed herein is intended to work in conjunction with a “standard” nebulizer, such as (but not limited to) the Hudson RCI 1724 Up-Draft Nebulizer.

Air exhaled by the patient is directed by the nebulizer into the bag.
(ii) a filter element (FE) may be disposed (on a wall of) the bag to allow air which is exhaled by the patient to escape into the environment, or into a collection reservoir (R);
   the filter, mounted over an opening on the sidewall of the bag, and the opening may be considered to be an “exhaust port”.
   the filter may be a flat, “pad” type filter (as may be contrasted with a canister type filter), and is intended to filter air exhaled by the patient, through the nebulizer, into the bag. A typical filter has three (3) layers. First layer absorbs the initial impact. Second layer intercepts the particles usually with an electrostatically charged fabric. Third layer uses diffusion to capture the low-mass particles. Reference https://www.vyaire.com/sites/us/files/2020-06/vyr-gb1-2000294-filter-considerations-in-the-covid-19-era-wp_final.pdf
   the filter may be supported by a flat, stiff (stiffener) element surrounding and supporting the filter itself, to allow the filter to be mounted, preferably air-tightly, to the wall of the bag. This may be somewhat akin to how a vacuum cleaner bag is supported by a stiff cardboard member.

Some additional components, or variations/modifications of the components (bag, filter) mentioned above may also be incorporated into the filtration device, and may be shown in subsequent pages.

For example, stiffening components (SC) may be added to portions of the bag to control its flexure during inhalation and exhalation, and to improve mounting the filtration device (FD) securely to the nebulizer exhaust tube (NET).

The bag is intended to be flexible, so that in use, when the patient inhales, the bag may collapse. The nebulizer may (or may not) have enough flow to permit this. Some nebulizers are not able to supply sufficient air flow and therefore the non-rebreather option (e.g., Page 3 embodiment) may not be usable. Conversely, the bag may expand when the patient exhales. When the bag collapses, the filter, or another component, may be drawn against the exhaust tube of the nebulizer, shutting it off.

The bag (B) may act like a bellows, and should be sufficiently large so that although the filter element (F, FE) is disposed in front of the inlet port (IP), the sponge element (S, AS) disposed on the nebulizer side of the filtration device (FD) prevents the filter (F) from acting like the aforementioned deflector (D). If the filter is ‘across’ the inlet port, then it may not move. It may abut the nebulizer exhalation tube (NET) thereby restricting the air flow to that portion of the filter element (F, FE). The addition (inclusion) of the sponge element (S, AS) between the filter element (F, FE) and the inlet port (IP) resolves that issue, allowing the filter element (F, FE) to move away from inlet port (IP) during exhalation.

A smaller device may be achieved by using a smaller, but thinner bag material, thereby increasing flexibility; however, it may make manufacturing more challenging.

In some embodiments, a flexible flap of material (not shown) may be disposed over the inlet port (IP), on the interior of the filtration device (FD), in a “normally closed” position, so that the flap (or filter element F), if disposed over the inlet port, may function as a one-way (“check”) valve that opens when the patient exhales through the nebulizer and into the filtration device (FD), and closes when the patient inhales.

It may be noted that some nebulizers have built in check valves, some do not.

Page 6

This page shows an embodiment of a non-rebreather exhalation filtration device (FD) with Aerosol Separator (AS) and collection reservoir (R).

It bears mention that the non-rebreather (e.g., “check valve”) feature is optional with various embodiments disclosed herein.

The left-hand illustration shows an additional deflector element (D).

The bag may be described as having two generally rectangular walls (the walls could be round), and may be manufactured from a single sheet of flexible plastic material having a lower (as viewed) generally rectangular portion (area) for one wall of the bag, and an upper (as viewed) generally rectangular portion (area) for the other wall of the bag.

A stiffener element (ST) (not shown in this embodiment) may be mounted to the lower portion of the bag, over an inlet port (IP).

The filter (F) may be mounted to the upper portion of the bag, over an outlet port.

A deflector element or component (D) may be incorporated into the bag of the filtration device (FD).

The deflector component (D) resides inside the bag, between the inlet port and the filter, so direct exhalation breath does not reach the filter but must travel around the deflector component (D) to exit through the filter.

The deflector component (D) should be rigid, is shown having a cross or cruciform shape, and may be disposed inside the bag between the inlet port and filter/outlet port as to not restrict the bag inflation/bellow movement when the patient exhales. To this end, the deflector component may be dimensioned so that it freely “floats” (is free to move towards or away from the inlet port), with a central portion covering/uncovering the inlet port, and it arms (extending radially from the central portion towards (but not connected to) respective side edges of the bag.

When the patient exhales, aerosols or particles suspended in the patient's breath will contacts the deflector, and may separate (from the gas in the patient's breath), and may collect in the bag. Refer to the diagram marked “Exhalation”.

When the patient inhales, the bag collapses under pressure and pushes the deflector onto the Handheld Nebulizer exhalation (exhaust) tube, blocking air from entry thereby forming a non-rebreather valve. Refer to the diagram marked “Inhalation”.

In this regard, the deflector component may function essentially as a check valve, allowing free flow in only one direction—into the bag from the inlet port, and out of the bag through the outlet port (through the filter).

Page 7

This page shows a Non-Rebreather Exhalation Filtration Device with Aerosol Separator and Collection Reservoir. See also Pages 8, 9.

This page shows a front view, a rear view and a side view of the filtration device (FD).

Also shown are
a side view with the filtration device mounted to a handheld nebulizer with mouthpiece.
a front view (perspective) of the filtration device mounted to a handheld nebulizer with mouthpiece.
a rear view (perspective) of the filtration device mounted to a handheld nebulizer with mouthpiece.

Page 8

This page shows Non-Rebreather Exhalation Filtration Device with Aerosol Separator and Collection Reservoir

This page shows a front view and a rear view of an embodiment of the filtration device (FD) with the outlet port disposed above the inlet port, on the same or on opposite sides of the bag.

Also shown are the following features, which may be associated with or incorporated into different embodiments of the filtration device (FD). These features may include (but are not limited to) the following.
The filtration device may be fabricated of a polyethylene plastic bag, or similar materials suitable to be formed into a bag (B).
The bag may be sealed (air-tight) around its periphery.
The bag has two cutouts (openings)—an inlet port (IP) and an outlet port (OP). These two ports (IP, OP) may be located on the same (front), or on opposite (front and back) sides (walls) of the bag.
A stiffener component (SC; see Page 9), such as an additional stiffer piece of plastic, may be disposed around the inlet port (IP) and/or the outlet port (OP) to ensure that they stay open, to ensure that the inlet port fits snugly onto the exhaust tube of the nebulizer, and the like. Generally, the filter element (F) mounted to the outlet port should maintain the integrity (size and shape) of the outlet port without the need for an additional stiffener.
A filter element (F, FE) is shown mounted to the outlet port, as mentioned above.
the inlet port may be undersized to create a tight seal with the nebulizer exhalation tube (NET).
the cutouts (for the inlet and outlet ports) may be on the same (wall) of the bag—i.e., the nebulizer side. The cutouts may extend only through one sheet (wall) of the bag, the other (opposite) wall not having any cutouts. For example, see FIG. 1, wherein the inlet and outlet ports are both disposed on the same (front, proximal) side of the bag, the opposite (rear, distal) side of the bag not having any holes, ports or cutouts in it.
As suggested above, the cutouts for the inlet and outlet ports do NOT have to be on the same side of the bag. They may be on opposite sides of the bag.

It is worth noting that the bottom of the bag (B) may be extended to form a collection reservoir (R), as mentioned above, for collecting aerosols and/or particles exhaled by the patient.

This page shows Non-Rebreather Exhalation Filtration Device with Aerosol Separator and Collection Reservoir

This page shows some alternative constructions, which may be considered to be “enhancements”.
As suggested above, a stiffener component (SC) may be added around the inlet port (IP) for increased rigidity while mounting the inlet port onto the nebulizer exhalation tube (NET). This stiffener component could be mounted either internally or externally to the bag (B).
A screen mesh may be disposed to reside inside the bag to separate/extract aerosol droplets out of the air stream thereby reducing filter dampness and increasing its effective duration. This screen mesh may function similarly to the above-described deflector component (D).
A collection Reservoir (R) may be disposed at the bottom of the bag to collect the extracted (i.e., separated by the screen mesh or deflector component) aerosol droplets.
Optionally, a desiccant (not shown) could be placed in the Collection Reservoir (R) to absorb the droplets (potentially contaminated bio-matter) to prevent their spillage through the ports when device is removed and discarded.

Notice that the filter element (F, FE) and outlet port (OP) are disposed in an upper portion of the filtration device (FD), above the lower portion of the filtration device (FD) which has the inlet port (IP).

The aerosol separator (AS) is disposed in the lower portion of the filtration device (FD), at the inlet port (IP), and may comprise a screen mesh (SM).

Also, a stiffener component (SC) is disposed around the inlet port (IP) for increased rigidity while mounting the inlet port (IP) onto the handheld nebulizer exhalation tube (NET). The stiffener component (SC) may be mounted internally or externally to the bag (B)

This page shows Non-Rebreather Exhalation Filtration Device with Aerosol Separator and Collection Reservoir

The upper illustration shows that during inhalation, the filtration device (FD) collapses to prevent flow from the environment.

The lower illustration shows that during exhalation, the filtration device (FD) opens to direct air from the handheld nebulizer exhalation tube (NET) through the filter element (F). Notice that the filter element and outlet port (OP) are disposed above the inlet port (IP).

Page 11 This page illustrates an embodiment of the filtration device (FD) adapted for in-line connection (attachment) to an evacuation hose or to a ventilation device.

In this embodiment, the filtration device (FD) may comprise:
a first plastic sheet (PS) disposed on the inlet side of the filtration device (FD), and having an inlet port (IP);
a second plastic sheet (PS2) disposed on the outlet side of the filtration device (FD), and having an outlet port (OP);
a filter element (F) disposed inside the filtration device (FD);
a first sponge element or component (AS1) disposed on the inlet side of the filtration device (FD), between the first plastic sheet (PS) and the filter element (F); and
a second sponge element or component (AS2) disposed on the outlet side of the filter, between the second plastic sheet (PS2) and the filter element (F).

Additionally,
an inlet tube (IT) may be disposed on or in the inlet port (IP)
an outlet tube (OT) may be disposed on or in the outlet port (OP)
a non-rebreather deflector (D or NRD, not shown) can be added at either the inlet port or outlet port side depending on intended flow direction.
dry air/oxygen gas flowing through the device can be humidified by soaking the sponge element(s) with warm water.

In some of the embodiments disclosed herein, an extra component, such as a tab, may be incorporated into the first plastic sheet to facilitate pulling the inlet port (IP) over the nebulizer exhaust tube (NET). Or, a stiffener, described below.

Page 12

This page shows three features of the sponge component(s) (AS, SE), and is applicable to embodiments with sponge component(s).

These features are described in the context of a nebulizer exhaust tube (NET) inserted into the inlet port (IP) of the filtration device (FD), such as was shown at Page 3.

Feature 1

This is shown in the two illustrations on the left

In the left one of these two illustrations (Restricted Flow Undesirable), it is illustrated that if the nebulizer exhaust tube (NET) is inserted into the inlet port (IP), without a sponge element (AS), the end of the nebulizer exhaust tube (NET) may abut the filter (F). This would be undesirable, as it would restrict flow through the filtration device (FD).

In the right one of these two illustrations (No Flow Restriction), it is shown that the sponge (AS) functions as a spacer (stand-off) between the end of the nebulizer exhaust tube (NET) and the filter element (F), avoiding contact between the two components which would cause a flow restriction, thus permitting unrestricted flow through the filtration device (FD).

Feature 2 This figure illustrates that the sponge supports the plastic sheet (inlet side) during insertion of the nebulizer exhaust tube (NET) into the inlet port of the filtration device (FD). This allows the inlet port (IP) to expand and “snap” over the nebulizer exhaust tube, and may eliminate a need for an extra component to pull the inlet port over the nebulizer exhaust tube.

Feature 3

This figure illustrates that the sponge acts as an aerosol extractor (or aerosol separator AS) which will keep the filter element (F, FE) dry, longer (than without the sponge “filtering” out aerosols). Aerosols may be extracted by the sponge prior to the air stream that they are in contacting the filter element.

As the aerosol travels through the many open cells of the sponge, the aerosol droplets bump into and merge with each other to form larger aerosol droplets that collect away from the filter, such as in a reservoir (R) portion of the bag (B). Some drops are illustrated at the bottom of the bag.

Page 13

This page shows an embodiment of a filtration device (FD) with only two components. Generally, the device is a two-dimensional assembly that conforms to allow an axial connection and forms a seal with the nebulizer exhaust tube (NET).

The figure(s) on the left are a side (cross-sectional) view and a front (inlet port side) plan view of the filtration device (FD), which may comprise:
a filter element (F), such as a disc shaped filter; and
a plastic sheet (PS) disposed over an inlet side of the filter element and having an inlet port (IP), such as for accepting the exhaust tube (NET) of a handheld nebulizer.

The figure(s) on the right are a side (cross-sectional) view and a front (inlet port side) plan view of the device, with the inlet port stretched over a tube which may be the nebulizer exhaust tube (NET) or an inlet tube (IT; refer to Page 11).

The inlet port (IP) may be sized to be smaller (in diameter) than the tube (NET or IT) so that a seal between the filtration device (FD) and the tube is formed at the interface of the inlet port (IP) in the plastic sheet and the tube. A seal may be formed at the interface, since the plastic inlet port may be undersized so that it stretches over the tube.

Both sets of figures show that the plastic sheet (PS) may be welded to the filter element (F) around their common peripheries (circumferences). This forms a type of bag (B), with one side being a plastic sheet (with an inlet port), the other side being the filter element which is permeable.

In a “relaxed” state (no airflow through the device), the substantially flat plastic sheet may be substantially abutting the substantially flat filter.

When air is passing into the device through the inlet port (IP) in the plastic sheet (and ultimately exiting through the filter), the “bag” tends to inflate, resulting in the plastic sheet and filter moving away from one another. This allows air entering the filtration device (FD), through the inlet port (IP), to self-distribute over the entire surface area of the filter element (F). No sponge or aerosol separator (AS) is shown in these figures, and may not be present in this embodiment.

Reference may be made to Feature 1 (Page 12) which discloses how the sponge element (aerosol separator (AS) prevents restricted flow, helps separate and protect the filter from aerosols, and may distribute incoming air over a greater area of the filter element (F, FE). The filter element (F, FE) has a much larger diameter than the inlet port (IP) or tube (NET, IT).

Page 14 This page illustrates Means of Facilitating Easy Mounting of Inlet Port Over Tube, Transforming the Two Dimensional Assembly (e.g., Page 13) into an Axial Connection.

The figure(s) on the left are a side (cross-sectional) view and a front (inlet port side) plan view of the device, and show that a filtration device (FD) may comprise:
a filter element (F), such as a disc shaped filter;
a plastic sheet (PS) disposed over the inlet side of the filter and having an inlet port (IP), such as for accepting the exhaust tube (NET) of a handheld nebulizer; and
a stiffener component (SC) attached around the inlet port to increase plastic sheet support around the inlet port so that the plastic sheet can be installed (such as pulled) over the tube (T) when the stiffener component is pulled towards the tube.

The stiffener (or stiffer) component (SC) may be an additional piece of plastic located around the inlet port (IP). The stiffener component may be in the form of a ring, having an inside diameter approximately equal to the diameter of the inlet port (IP), and an outside diameter greater than the inside diameter and less than the outside diameter of the plastic sheet (PS) or the filter element (F).

Again, reference may be made to Feature 1 (Page 12) which describes how incorporating a sponge element (aerosol separator (AS)) may be useful.

The figure(s) on the right are a side (cross-sectional) view and a front (inlet port side) plan view of the device, and show that a filtration device (FD) may comprise:
a filter element (F,FE), such as a disc shaped filter;
a plastic sheet (PS) disposed over the inlet side of the filter element and having an inlet port (IP), such as for accepting the exhaust tube (NET) of a handheld nebulizer; and
a sponge (AS) disposed between the plastic sheet and the filter.

The sponge supports the plastic sheet (PS) during tube insertion so that the inlet port (IP) can expand and fit snugly over the tube.

It should be understood that various features or elements of the various embodiments disclosed herein can be “mixed and matched” to produce a wide variety of filter devices suitable for various applications. All of the embodiments are intended to be low cost, effective, and disposable (single use). Pages 15-23 disclose some additional embodiments and/or variations of the filtration device (FD).

This page (“Means of Mounting of Filter Inlet Port Onto a Flat Surface”) illustrates an embodiment of a filtration device (FD) and a technique for mounting the filtration device (FD) onto a flat surface, such as the flat surface of a device with a port that emits aerosol such as a nebulizing therapy mask or an oxygen therapy mask (M). Refer also to FIGS. 5A,B (which are similar to FIGS. 3A,B). The mask (M) is not part of the filtration device (FD), per se.

The filtration device (FD) is similar to the filtration device (FD) shown at Page 14, but the filtration device (FD) is not designed to fit (slide) onto a tube, such as a nebulizer exhaust tube (NET), with interference fit etc.. Rather, the filtration device (FD) is adapted to be mounted (adhered) to the mask (M), by lining up the inlet port (IP) of the filtration device (FD) with a port (P) of the mask (M) and sticking (adhesively mounting) the filtration device (FD) to the mask (M). This works well with a mask (M) having its port (P) on a flat portion of the mask (M).

To effect this mounting technique, a layer of adhesive (AL) may be disposed around the inlet port (IP) of the filtration device (FD). The layer of adhesive may be applied directly to the front surface of the plastic sheet (PS) of the filtration device (FD), around the inlet port (IP).

The layer of adhesive (AL) may be applied to the front plastic sheet (PS) of the filtration device (FD) immediately before assembling the filtration device (FD) to the mask (M). Alternatively, the layer of adhesive (AL) may be applied to the filtration device (FD), and protected by a protective release layer (RL) which may be removed immediately before assembling the filtration device (FD) to the mask. The release layer (RL) may comprise a thin (0.005 in; 0.125 mm thick) layer of paper or plastic liner coated with a silicone release agent, disposed on the exposed (other, opposite, mask) side of the adhesive layer (AL) or element.

The adhesive layer (AL) may be sprayed or “painted” onto the plastic sheet (PS) at the front (mask side) of the filtration device (FD). Alternatively, the adhesive layer (AL) may comprise a thin 2 mil (0.002 in; 0.05 mm) layer of pressure-sensitive adhesive, which is affixed on one side to the plastic sheet (PS) of the filtration device (FD).

The filtration device (FD) may comprise the following elements (components):
PS plastic sheet
IP inlet port
AL adhesive layer (aka “stickie” interface SI)
FE filter element
SE sponge element

The upper (cross-sectional view) illustration on the left-hand side of the page corresponds with FIG. 5A, and shows an embodiment of the filtration device (FD), comprising:
a plastic sheet (PS) having a periphery and an inlet port (IP);
a filter element (FE) disposed behind (to the right of, as viewed) the plastic sheet, and attached (such as welded) around its periphery to the periphery (or another area) of the plastic sheet at its back surface;
a sponge element (SE) disposed between the plastic sheet and the filter element; and
an adhesive layer (AL) or “stickie” interface (SI) disposed on a front surface of the plastic sheet (PS), around the inlet port (IP). (The AL is shown “exploded” away from the PS.)

The lower (plan view) illustration on the left-hand side of the page corresponds with FIG. 5B, and shows the filter (F), and the plastic sheet, filter element, sponge element, and stickie” interface.

The illustration in the middle of the page shows the filtration device (FD) positioned to be attached (mounted) to a mask (M) having a port (P). To mount the filtration device (FD) to the mask, a user may simply push the filtration device (FD) onto the mask (M).

The illustration on the right-hand side of the page shows the filtration device (FD) attached (mounted) to the mask (M). The “stickie” interface, or adhesive layer (AL) is for mounting the filtration device to the mask, and removably holds the filtration device (FD) in place on the mask with the inlet port (IP) of the filtration device (FD) aligned with the port (P) of the mask, and also forms a seal between the filtration device (FD) and the mask.

In this embodiment of a filtration device (FD), the surface of a mask (M) to which the filtration device (FD) is attached is flat, and it may also be rigid. This embodiment is suitable for use with a mask having its port disposed on a flat, rigid area of the mask. Not all masks will have such a flat, rigid area, and the next two pages (Pages 16, 17) disclose an embodiment of the filtration device (FD) adapted for use with masks that do not have their port disposed on a flat, rigid area of the mask.

Page 16

This page (“Means of Mounting of Filter Inlet Port Onto a Non-Flat Flexible Surface”) illustrates a problem which may be encountered with mounting the filtration device (FD) shown at Page 15 to a non-flat or flexible surface of a mask (M). As illustrated, when the surface of the mask flexes, stresses may be induced at the interface between the front plastic sheet (PS) of the filtration device (FD) and the mask, which may cause the seal at the interface to break and consequent separation of the filtration device (FD) from the mask.

Page 17

This page (“Means of Mounting of Filter Inlet Port Onto a Non-Flat Surface”) illustrates a technique for mounting the filter onto a non-flat surface, such as the non-flat surface of a mask.

See also FIGS. 6A,B. The following elements of the filtration device (FD) are shown:
PS plastic sheet
IP inlet port
FS flexible substrate
AL adhesive layer
FE filter element
SE sponge element

The filtration device (FD) is similar to the filtration device (FD) shown at Page 15, but in this embodiment the interface between the filtration device (FD) and the mask (M) comprises a thin (such as 0.006 in; 0.152 mm) flexible substrate (FS), or sheet of a plastic material (such as polyethylene), which may be ring shaped (having an inside diameter and an outside diameter) and affixed (such as welded or adhesively secured) at its inner diameter (or on one side of the flexible sheet) to the plastic sheet (PS) of the filtration device (FD), at the inlet port (IP). An adhesive layer (AL) may be disposed on the mask side of the flexible substrate (FS) for mounting the filtration device (FD) to the mask (M) having a non-rigid or non-flat mounting surface.

The flexible substrate (FS) may be donut-shaped, having an overall outer diameter (OD) and an opening or inner diameter (ID). The flexible substrate may be secured to the front surface of the plastic sheet (PS), with its opening aligned with the inlet port (IP), by welding (or adhesively sticking) the flexible substrate to the plastic sheet, around the inlet port.

The flexible substrate (FS) may be adhered such as by welding to the filtration device (FD), with the central opening of the flexible substrate aligned with the inlet port (IP) of the filtration device (FD). The filtration device (FD), may then be mounted/adhered to the mask, by lining up the inlet port (IP) of the filtration device (FD), with the mask port (P), and sticking the filtration device (FD) to the mask.

The flexible substrate (FS) may first be adhered to the filtration device (FD), with a release layer (RL) protecting the adhesive on the mask side of the flexible substrate (FS). Adhesive on the mask side of the flexible substrate (FS) may be protected by a release layer (as discussed at page 15) which may be removed immediately before assembling (mounting) the filtration device (FD), to the mask (M). In this case, the flexible substrate (FS) and release layer (RL) may be considered to be part of, or a component of the filtration device (FD).

Alternatively, the flexible substrate (FS) could first be adhered to the mask, thereafter the filtration device (FD) being assembled to the mask (M). In this case, a release layer (RL) may be provided on the filtration device (FD) side of the flexible substrate (FS). In this case, the flexible substrate (FS) may be considered to be part of the mask (M).

Alternatively, the flexible substrate may not first be adhered to either the filtration device (FD) or to the mask (M). It may simply be donut shaped, with adhesive on both sides thereof, and a release film on both sides thereof protecting the adhesive, akin to double-sided tape. Then, when assembling the filter to the mask, the release layers on both (filter, mask) sides of the flexible substrate may be removed. In this case, the flexible substrate (FS) may be considered to be associated with the filtration device (FD), although it may be supplied separately therefrom.

Adhesive on the filter side and on the mask side of the flexible substrate (FS) may cover (extend over) only select areas of the flexible substrate radially outward from the central opening in the flexible substrate. For example, on the filter side of the flexible substrate, adhesive may be disposed only on a radially inward portion of the flexible substrate. However, as mentioned above, the flexible substrate may simply and suitably be welded to the front plastic sheet (PS) of the filtration device (FD), at its inlet port (IP). And, on the mask side of the flexible substrate, adhesive may be disposed only on a radially outward portion of the flexible substrate. In other words, the portions of the flexible substrate joined with the filtration device (FD) and the mask (M) may be radially offset from one another, in a sort of cantilever fashion, to allow for the flexible substrate (FS) to flex without compromising the integrity of the adhesive seal between the filtration device (FD) and the mask (M).

It should be noted that the flexible substrate (FS) may be other than donut shaped, and the opening therein may be located other than centrally on the flexible substrate.

The upper (cross-sectional view) illustration on the left-hand side of the page corresponds with FIG. 6A, and shows an embodiment of the filtration device (FD), comprising:
a plastic sheet (PS) having a periphery and an inlet port (IP);
a filter element (FE) disposed behind (to the right of, as viewed) the plastic sheet, and attached (such as welded) around its periphery to the periphery (or another area) of the plastic sheet at its back surface;
a sponge element (SE) disposed between the plastic sheet and the filter element; and
a flexible substrate (FS) carrying a “stickie” interface (SI) mounted to the plastic sheet (PS).

In the embodiments shown in FIGS. 3, 4, 5 (A,B) and 6 (A,B), filter element (FE) and front plastic sheet (PS) create a sort of bag, comparable to the bag (B) described above with respect to FIGS. 1 and 2, and an area (space, volume) inside of the filtration device (FD) below the sponge element (SE) or aerosol separator (SE) may function as a reservoir (R) for collecting aerosol droplets and the like which may be separated from the air exhaled by the patient through the filtration device (FD) by the aerosol separator (AS) or sponge element (SE)

The lower (plan view) illustration on the left-hand side of the page corresponds with FIG. 6B, and shows the filtration device (FD), and the plastic sheet, filter element, sponge element, and stickie” interface.

The illustration in the middle of the page shows the filter positioned to be attached (mounted) to a mask (M) having a port (P). To mount the filter to the mask, a user may simply push the filter onto the mask.

The illustration on the right-hand side of the page shows the filter attached (mounted) to the mask. The “stickie” interface holds the filter in place on the mask, with the inlet ort of the filter aligned with the port of the mask, and forms a seal between the filter and the mask.

Note that, in this embodiment, the mask surface to which the filter is attached in not flat, and it may also not be rigid.

In some embodiments disclosed herein, the use of an adhesive layer (AL) or “stickie” interface (SI) for mounting/attaching) the filtration device (FD) to a mask (M) has been described. A suitable adhesive which can be use for this purpose is 3M 483 Polyethylene Film Tape, is made with same material as the plastic sheet (PS) for bonding compatibility, is stretchable, and the adhesive likes to adhere to surfaces which masks are typically fabricated with, such as vinyl.

This embodiment is suitable for use with a mask having its port disposed on a non-flat and/or flexible area of the mask.

This page illustrates some components of an embodiment of the filtration device (FD). In this embodiment, the inlet port (IP) is not concentric with the filter media or element (F, FE).

The illustration on the left-hand side of the page shows (plan view) the following components, individually, prior to assembly of the filtration device (FD):
a filter media (FM), or filter element (FE);
a sponge element (SE);
a plastic sheet (PS) which serves as the mask side of the overall filtration device (FD);
an inlet port (IP), or opening in the plastic sheet; and
a pull tab (PT), serving as the aforementioned release layer (RL) which protects a generally donut shaped flexible “stickie” substrate (SS; obscured by the pull tab and not visible in this view).

The filtration device (FD), may be round, more accurately circular or disc-like. Similarly, the plastic sheet (PS), filter media (FM) and sponge element (SE) may all be round. the filter media may have a diameter approximately equal to the diameter of the plastic sheet, such as 4 inches (10 cm). The filtration device (FD) and its individual components may be other than round, such as elliptical, triangular, square, rectangular, or polygon shaped (where the number of sides of the polygon is greater than 4, such as 5, 6 or 8). Other shapes are possible.

In this embodiment, the inlet port of the filtration device (FD) is off-center (or non-concentric) with the circular (disc-like) plastic sheet (PS), filter media (F), and sponge element (S).

The illustration on the right-hand side of the page shows (2 plan views) the “stickie” substrate (SS) and pull tab (PT).

The “stickie” substrate (SS) is shown with a pull tab (PT) and optional protective cover (PC, or release layer RL) over glue (adhesive, AL) on the stickie substrate for protection prior to use. A pull tab cover (PTC) is also shown.

The pull tab (PT) is only the tab portion of the sticky sheet (SS) and optional protective cover (PC). The pull tab cover (PTC) is a separate piece that masks (separates, isolates) the extra adhesive on the tab so the tab on the sticky sheet (SS) and protective cover (PC) are not connected. Easy separation by the user and avoids contamination of the user finger(s) during mounting.

The pull tab (PT) is useful for easy removal of the pull tab cover; plus the removal of the filter assembly from the mask.

The pull tab (PT) allows pulling the filtration device (FD) off the mask (M). In some instances where patients require extended treatments or repeated treatments, saturated filters can be removed and a new filtration device (FD) mounted to eliminate the need to discard the mask.

The pull tab cover (PTC) provides a stickie barrier on the Pull Tab section, so the Pull Tab does not adhere to the mask surface or the user's fingers during mask application.

This comports with the filter embodiment described at Page 17, for a mask having a non-flat surface for attachment of the filter.

The Page 17 embodiment would work on a flat surface also, but complies substantially better with non-flat surfaces than the Page 15 embodiment.

Page 19

This page illustrates the plastic sheet (PS) and “stickie” substrate (SS) components of an embodiment of the filtration device (FD), comporting with the embodiment shown and described at page 17.

In the illustration on the left-hand side of the page, the “stickie” substrate (SS) is shown on the exterior (mask-facing) side of the plastic sheet (PS), the opening in the “stickie” substrate (SS) being aligned with the inlet port (IP) of the filtration device (FD) in the plastic sheet. These openings may be, as shown, off center with the circular plastic sheet (PS), such as asymmetrically biased towards the top portion of the filtration device (FD). This is to allow for the mounting of filtration devices (FD) having larger filter elements (FE) onto masks. The offset allows the filtration devices (FD) to reside toward the front of the mask and further from the back the mask (face interface side) so they do not contact the patient's face when the mask is on the patient.

In the illustration on the right-hand side of the page, the protective cover (PC) is shown pulled back to expose a weld line between the plastic sheet (PS) and the stickie substrate (SS).

Page 20

This page illustrates an embodiment of the filtration device (FD), and shares some features with the embodiment(s) disclosed at Pages 17, 18, 19, hereinabove.

The illustrations on the left-hand side of the page are (i) a plan view (upper illustration) of the filtration device (FD) and (ii) a cross-sectional view (lower illustration) taken on a line A-A through the plan view (upper illustration).

The illustration on the right-hand side of the page is a close-up or detailed cross-sectional view of a portion of the filtration device (FD) shown in the lower illustration on the left-hand side of the page showing the plastic sheet, stickie (flexible) substrate, a weld line where the flexible substrate is welded (joined) to the plastic sheet, and the pull tab.

Page 21

This page illustrates a common respiratory mask (M) with open ports. the close-up view on the right-hand side of the page illustrates that the area around the port on the mask is not flat. The filtration device (FD) embodiment(s) described above at Pages 17, 18, 19, 20 are suitable to be mounted (attached) to such a mask.

Page 22

This page illustrates a filter (or filter assembly), such as described above at Pages 17, 18, 19, 20 mounted to a common respiratory mask (with open ports), such as was shown at page 21.

The mask surfaces around the port cutouts could be flat or curvy (non-flat). Pages 22 and 23 show the same mask from various angles to demonstrate how the embodiment of Pages 17, 18, 19, 20 complies with the mask surface curvature around the ports to create a reliable seal.

The illustration on the left-hand side of the page is an external (front) mask view, showing the filter assembly mounted to the mask.

The illustration on the right-hand side of the page is an internal (rear) mask view, showing the filter assembly mounted to the mask and, more particularly, the interface seal of the filter to the mask.

Page 23

This page illustrates a filter (or filter assembly), such as described above, mounted to a common respiratory mask having a port on a non-flat surface.

The illustration on the left-hand side of the page shows the filter assembly (filtration device), and the flexible (stickie) substrate with pull tab.

The illustration on the right-hand side of the page shows that the flexible (stickie) substrate allows the filtration device to be mounted compliantly to a face mask having a non-flat surface.

### Some Additional Embodiments

FIG. 5A of parent U.S. Ser. No. 17/734,133 shows providing an adhesive layer (AL, or sticky interface) directly on the plastic sheet (PS) of the filtration device (FD) for attaching the filtration device (FD) to a facemask (not shown in the figure, refer to APPENDIX 1, pages 15 et seq). This technique for attaching the filtration device (FD) to the facemask may suffer from delamination (de-adhesion) due to factors such as temperature and stress, particularly when the filtration device is stored.

FIG. 6A of parent U.S. Ser. No. 17/734,133 shows incorporating a flexible substrate (FS) between the plastic sheet (PS) of the filtration device (FD) and the facemask (not shown in the figure, refer to APPENDIX 1, pages 15 et seq). This technique reduces the problem of delamination (de-adhesion), but may also be compromised due to factors such as temperature and stress, particularly when the filtration device is stored.

The FIGS. 5 (A,B) and 6 (A,B) embodiments disclose having a “stickie” interface between the filtration device (FD) and the facemask (M). In other words, the method of mounting the filtration device (FD) to the facemask (M) relies on an adhesive, which may degrade over time, including when the filtration device (FD) is in extended use or in storage.

Some of the embodiments described hereinbelow disclose having a more reliable mechanical interface between the filtration device (FD) and the facemask (M), such as wherein each of the filtration device (FD) and facemask (M) are provided with one half of a pair of mating elements (IE and EE, shown generically in FIGS. 7A,B, and described in greater detail hereinbelow) which may be connected with one another, such as by screwing the filtration device (FD) onto the facemask (M).

U.S. Pat. No. 7,559,323 (2009-07-14; Hacke et al.) discloses a disposable mask assembly with exhaust filter. A method of assembling a face mask is provided for a patient that includes a face piece sized to fit over the patient's nose and mouth. The face mask assembly forms a mask chamber between the face piece and the patient's nose and mouth. An inhalation adapter is coupled to the face piece to deliver medication to the chamber. A filter housing is coupled to the face piece and includes a flange section that defines a passageway to connect the mask chamber and the flange section. A filter is positioned in the filter housing. A cover is coupled to the flange section and has an exhalation opening or vent to allow gases from the mask chamber to pass through the filter and escape from the passageway to the atmosphere. Hacke further discloses,
As best seen in FIGS. 1-3, the face piece 20 preferably comprises a substantially curvilinear shaped shell configured to fit over the nose and mouth of a patient to establish a seal. As shown in FIG. 2, a breathing mask chamber 22 is formed between an inner surface of the face piece 20 and the nose and mouth of the patient. Preferably, a lower portion of the face piece is provided with an inhalation aperture 23. The inhalation aperture 23 enables a supply of air to travel into the mask chamber 22 from the inhalation adapter 30. Apertures 24 and 26 extend through the face piece 20 to receive a neck portion 42 (FIG. 3) of the two filter housings 40 via a snap fit. The apertures 24 and 26 fluidly connect the mask chamber 22 with a passageway 44 (FIG. 3) of the two filter housings 40. The apertures 24 and 26 preferably are substantially circular in shape; however, other suitable shapes and sizes may be used.

US 2021/0330912 (2021-10-28; Groman et al.) discloses an adapter for retrofitting filters to face masks. Filters welded to the mask. A spacer allowing a check valve to operate. A method of providing oxygen and/or a nebulizing treatment to a patient is disclosed. Modifications to a mask may include: adding nose cushion to increase conformance of the mask to facial features; covering breathing ports with viral/bacterial filters; and adding desiccant material to capture moisture accumulation. A face plate fitted over and working in conjunction with a mask which pushes the mask against a user's facial features to assure a better fit. Straps are disposed on the face plate, rather than on the mask. The face plate may provide or comprise: filter protection from user contact, strap locking features, and a nebulizer cutout. As disclosed therein,
FIG. 4 illustrates (diagrammatically) a mask, fitted with a check valve (in an opening), a filter which may be disposed over the opening, and a spacer such as described above fitted over the opening between the filter and the face mask. The spacer allows the valve to function (i.e., to deflect outwards), unimpaired by the filter.

U.S. Pat. No. 7,559,323 (2009-07-14; Hacke et al.) discloses a face mask assembly and method of assembling a face mask is provided for a patient that includes a face piece sized to fit over the patient's nose and mouth. The face mask assembly forms a mask chamber between the face piece and the patient's nose and mouth. An inhalation adapter is coupled to the face piece to deliver medication to the chamber. A filter housing is coupled to the face piece and includes a flange section that defines a passageway to connect the mask chamber and the flange section. A filter is positioned in the filter housing. A cover is coupled to the flange section and has an exhalation opening or vent to allow gases from the mask chamber to pass through the filter and escape from the passageway to the atmosphere.

According to an embodiment of the invention, a technique for mounting the filtration device (FD) to the plastic sheet (PS) of a facemask is provided which may be more durable than the aforementioned (FIGS. 5A, 6A) adhesive techniques. Generally, a secure “mechanical” (rather than adhesive) solution is provided which may involve an external (to the facemask) piece, component or element (EE) mounted to the filtration device (FD) and an internal (to the facemask) piece, component or element (IE) suitable to attach securely (mechanically) to the external piece (EE) when the filtration device (FD) is mounted to the facemask.

Some examples of the mating external (EE) and internal (IE) elements will now be discussed.

FIGS. 7A and 7B are diagrams (cross-sectional views) illustrating, some methods of and apparatus for effecting a robust, reliable connection between the filtration device (FD) and a facemask (M), according to some embodiments of the invention.

FIG. 7 shows a portion of the plastic sheet (PS) of a filtration device (FD). An inlet port (IP) is provided through the plastic sheet (PS). The sponge element (SE) and filter element (FE) of the filtration device (FD) are omitted, for illustrative clarity, but would be disposed to the right (as viewed) of the plastic sheet (PS) as they were in FIG. 5A. Also shown is a portion of a facemask (M) having a mask port (P, or MP). The facemask (M) is shown spaced apart from the plastic sheet (PS) of the filtration device (FD), for illustrative clarity. When the filtration device (FD) is mounted to the facemask (M), the inlet port (IP) of the filtration device (FD) is aligned (dashed lines) with the mask port (MP) of the facemask (M) and the facemask (M) abuts the plastic sheet (PS) of the filtration device (FD).

FIG. 7A shows (i) an internal element (IE) positioned to be disposed on an internal surface of the facemask (M), with an opening (dashed lines) in the internal element aligned with the opening (MP) in the mask, and (ii) an external element (EE) positioned to be disposed on an internal surface of the filtration device (FD), with an opening (dashed lines) in the external element aligned with the inlet port (IP) of the filtration device (FD). The internal (IE) and external (EE) elements may be disc-shaped with an opening, or annular.

The external element (EE) may be permanently affixed to the plastic sheet (PS) of the filtration device, on the inside surface of the plastic sheet (as shown in FIG. 7A), or on the external surface of the plastic sheet (as shown in FIG. 7B)

In use, the internal element (IE) may be positioned over the mask port (MP) of the facemask (M). The filtration device (FD) would then be presented to the facemask, or vice-versa, and the internal element (IE) and mating external element (EE) may be joined together with the area of the facemask (M) having the mask port (MP) captured between the internal element (IE) and the external element (EE).

In the FIG. 7A version, the plastic sheet (PS) of the filtration device (FD) is also captured between the internal element (IE) on the interior surface of the facemask (M) and the external element (EE) on the interior surface of the plastic sheet (PS). and the external element (EE). In the FIG. 7B version, the plastic sheet (PS) of the filtration device (FD) is mounted to the facemask by the external element (EE) on the exterior surface of the plastic sheet (PS) and the internal element (IE) on the interior surface of the facemask (M). In either case, the object is to securely mount the filtration device (FD) to the facemask (M) with the inlet port (IP) of the filtration device (FD) aligned and in fluid communication with the mask port (MP) of the facemask.

The sponge element (SE, SP) and filter element (FE) are omitted from the views of FIGS. 7A, B for illustrative clarity.

When the internal element (IE) and external element (EE) are threaded (one having internal threads, the other having external threads), said mounting of the filtration device (FD) to the facemask (M) may be effected by introducing the filtration device (FD) to the facemask (M), with the inlet port (IP) and mask port (MP) aligned with one another, and either (i) rotating the internal element (IE) associated with the facemask (M) while holding the filtration device (FD) with its external element (EE) stationary, or (ii) rotating the filtration device (FD) with its external element (EE) while holding stationary the internal element (IE) associated with the facemask (M), or (iii) rotating both the internal element (IE) and the external element (EE) joined to the plastic sheet of the filtration device (FD).

With such an exemplary “threaded” joining of the filtration device (FD) to the facemask (M), the filtration device (FD) may also readily be removed from the facemask (M), and another filtration device (FD) installed onto the facemask (M).

The internal (IE) and external (EE) elements are shown “generically” in FIGS. 7A,B. These two elements may be generally disc shaped (with an opening), and may be made of plastic. Various means for joining the internal (IE) and external (EE) elements may be provided, such as (i) internal threads on one of the elements and external threads on the other of the elements, (ii) one of the elements having an annular lip and the other of the elements having an annular recess or trough for receiving the lip, (iii) the two elements joining with one another via a “bayonet” type interface, and (iv) any suitable means for joining the two elements mechanically with one another (exclusive of adhesive).

The external element (EE) may be a mounting nut (N) having internal threads, and is illustrated in greater detail in Appendix-3.

Appendix-2

Included with this filing is a document (Appendix-2), which is incorporated by reference herein and forms part of the Specification (description) hereof. This document comprises 4 pages. This document contains text. Some changes or “editorial comments” may be presented herein. This document contains illustrations, which may be considered to be “Drawings” (or Figures of the Drawing), some of which correspond to drawing figures presented herein.

Page 1

This page shows an embodiment of a rigid mounting scheme, with Internal and External Elements (IE and EE), and without the “stickie” (adhesive mounting), such as was shown generally in FIG. 7 (A,B).

On the left side of the page may be seen (in the box) an illustration of the embodiment (BG-131 embodiment) where the filtration device (FD) is attached to the facemask (M) with an adhesive layer (AL) forming a “stickie” interface, such as has been shown in FIG. 5A or 6A.

To the right of that “BG-131” illustration is an image showing an embodiment where the “stickie” (adhesive layer; AL) is done away with, and replaced by the external element (EE) described hereinabove. Here, the external element (EE) is shown on the inside surface of the plastic sheet (PS), such as was shown in FIG. 7A.

The external element (EE) associated with the filtration device (FD) may be a hollow tubular (cylindrical) element having external threads. The external element (EE) may alternatively be disposed on the internal surface of the sponge element (SE). Being hollow tubular, the external element (EE) may constitute (serve as) the inlet port (IP) of the filtration device.

Below the image showing the external element (EE) is an image of the internal element (IE) which would be disposed on an internal surface of the facemask (M), as described above.

The internal element (IE) associated with the facemask (M) may be a hollow tubular (cylindrical) element having internal threads.

Alternatively, the internal element (IE) may have external threads, and the external element (EE) may have internal threads.

In use, the filtration device (FD) may be mounted to the facemask (M) by screwing the filtration device (FD) with its external element (EE) onto the facemask (M) with its internal element (IE). In other words, the external element (EE) “mates” with the internal element (IE) when the filtration device (FD) is mounted to the facemask (M).

Each of the internal and external elements (IE and EE, respectively) may have a “flange” or disc-like coaxial extension of its hollow tubular portion of the element to permit secure mounting over an increased surface area to an internal surface of the facemask (M) or filtration device (FD), respectively. The image of the internal element (IE) shows the flange very well. The external element (EE) may be permanently affixed to the filtration device (FD), such as to the inner surface of the plastic sheet (PS).

FIGS. 8A and 8B show the internal (IE) and external (EE) elements with flanges. In this example the internal element (IE) associated with the facemask (M) has internal threads, and the external element (EE) associated with the filtration device (FD) has external threads. The internal and external elements may be made of molded plastic. The external element (EE) may be glued or welded to an appropriate portion (such as the plastic sheet PS) of the filtration device (FD) but could also be free floating since it is compressed against the Plastic Sheet (PS) to form a seal when the external element (EE) and internal element (IE) are joined. The internal element (IE) may be provided with the filtration device (FD), temporarily screwed onto the external element (EE), with a cardboard piece inserted therebetween with instructions and/or illustrations of how to mount the filtration device (FD) to the facemask (M).

The illustration on the upper right portion of the page shows a filtration device (FD) with the tubular portion of its external element (EE) extending through the port (MP, P) of the facemask (M) and the internal element (IE) awaiting mating with the external element (EE).

The illustration on the lower right portion of the page shows the filtration device (FD) with the tubular portion of its external element (EE) extending through the port (MP, P) of the facemask (M) with the internal element (IE) screwed onto (mated with) the external element (EE).

These two illustrations on the upper and lower right portion of the page are views taken from the interior of the facemask (M).

Page 2

This page shows another embodiment of a rigid mounting scheme, in this example with a rigid support (RS) which functions as an external element (EE), and which eliminates the plastic sheet (PS). As with the embodiment shown on the previous page, the “stickie” (adhesive mounting) has also been eliminated.

On the left side of the page may be seen (in the box) an illustration of the embodiment (BG-131 embodiment) where the filtration device (FD) is attached to the facemask (M) with an adhesive layer (AL) forming a “stickie” interface, such as has been shown in FIG. 5A or 6A. This is a repeat of the corresponding illustration on the previous page.

To the right of that “BG-131” illustration are images showing an embodiment of the filtration device (FD) which does not use the plastic sheet (PS).

In this embodiment, the rigid support (RS), which functions as the external element (EE), has a flange (FL) which is substantially the same size (diameter) as the filter element (FE). The sponge element (SE) may be disposed behind the oversize flange of the rigid support (RS), may free float or may be joined (such as by welding or gluing) to the rigid support (RS). The filter element (FE) may be disposed behind the sponge element (SE) and may be joined (such as by welding or gluing) to the rigid support (RS).

FIG. 9 is illustrative of this embodiment having a rigid support (RS). The rigid support (RS) may be a molded plastic piece, and functions as the external element (EE) associated with the filtration device (FE) for mounting the filtration device (FD) to the facemask (M) by screwing the externally-threaded rigid support (RS) into the internal element (IE) associated with the facemask (M). The rigid support (RS) eliminates the need for the plastic sheet (PS). The internal element (IE) associated with the facemask (M) may be similar or identical to the internal element (IE) shown in and described with respect to FIG. 8A

The internal element (IE) associated with the facemask is shown in the illustrations to the right of the “BG-131 embodiment”. The upper illustration shows the filtration device (FD) as viewed from the inlet side, wherein the rigid support (RS) with its externally-threaded tubular portion is visible. The lower illustration shows the filtration device (FD) as viewed from the outlet side, wherein the filter element (FE) is visible and the rigid support (RS) is not visible.

The illustrations on the right-hand portion of the page show the filtration device (FD) with rigid support (RS) mounted to a facemask (M).

The illustration on the upper right portion of the page shows the filtration device (FD) with the tubular portion of its rigid support (RS) extending through the port (MP, P) of the facemask (M) and the internal element (IE) awaiting mating with the external element (EE).

The illustration on the lower right portion of the page shows the filtration device (FD) with the tubular portion of its rigid support (RS) extending through the port (MP, P) of the facemask (M) with the internal element (IE) screwed onto (mated with) the external element (EE).

These two illustrations on the upper and lower right portion of the page are views taken from the interior of the facemask (M).

FIG. 9A shows a variation of the FIG. 9 embodiment of a rigid support (RS) wherein the rigid support (RS) has a flat flange (outer annular portion) In this variation of the FIG. 9 embodiment, the sponge element (SE) is smaller in diameter than the rigid support (RS), and the filter element (FE) “wraps around” the periphery of the sponge element (SE) and may be affixed (such as by welding) to an outer portion, such as an outer peripheral annular region of the rigid support (RS), on the outlet side thereof. In this figure, the filter element (FE) is shown as (represented by) a thick gray line (rather than the cross-hatching used in FIGS. 9 and 9B).

FIG. 9B shows a variation of the FIG. 9 embodiment of a rigid support (RS) wherein the rigid support (RS) has a flange (outer annular region) with sides. In this variation of the FIG. 9 embodiment, the outlet side of the rigid support (RS) is recessed to accept the sponge element (SE) in an inner region thereof. An outer peripheral annular region of the rigid support (RS), on the outlet side thereof, is not recessed. The sponge element (SE), having a diameter less than that of the rigid support (RS), may reside “flush” in the inner recessed region of the rigid support (RS), on the outlet side thereof, and the filter element (FE) may extend substantially flat across the sponge element (SE), on the outlet side thereof, and may be affixed (such as by welding) to the outer annular region of the rigid support (RS).

In the examples of FIG. 9A ad 9B, the sponge element (SE) may be, for example, 10-20 mm smaller (in diameter) than the rigid support (RS), leaving at least a 3-4 mm peripheral (outer) annular region of the rigid support (RS) for a corresponding peripheral (outer) annular region of the filter element (FE) to be affixed to said peripheral (outer) annular region of the rigid support (RS). Although these elements (RS, SE, FE) have been described as being circular, they may be other than circular, such as elliptical, etc.

The rigid support (RD) with flat flange (FIG. 9A) is essentially an oversize external element (EE), obviating the need for (performing the function of) the plastic sheet (PS). Compare FIGS. 7A,B. In the example (FIG. 9B) of the rigid support (RS) has side walls around its periphery, forming a recess for receiving (or encapsulating) some or all (depending on the depth of the recess) of the sponge element (SE). With the filter element (FE) disposed on the outlet side of the sponge element (SE), the sponge element may be compressed, as may be desired, in one or both of the axial and radial directions. The embodiment of FIG. 9B, with a wall on the flange, may provide for easier component assembly, particularly with small filter surfaces, since the recess (FIG. 9B) will “automatically” properly locate (position) the sponge element (SE). Generally, all of the components (rigid support RS, sponge element SE, filter element FE) should be coaxial, and the inlet port (IP) may also be coaxial with the other components. Although these components (RS, SE, FE) have been described as circular, they may have another shape such as elliptical, etc.

Page 3

The illustration on the top left of this page shows the rigid support (RS) with flat flange, as was illustrated in FIG. 9A.

Below that is a view of the inner surface of the rigid support (RS) with the sponge element (SE) disposed on the inner surface of the rigid support (RS). As shown, and as was described with respect to FIG. 9A, the sponge element (SE) has a smaller diameter than the rigid support (RS). The rigid support (RS) has a flat outer (peripheral) annular region to which the filter element (FE; not shown here) may be affixed.

The illustration on the top right of this page shows the rigid support (RS) recessed on its outlet side leaving a flange with walls, as was illustrated in FIG. 9B.

Below that is a view of the inner surface of the rigid support (RS) with the sponge element (SE) disposed in the recess on the inner surface of the rigid support (RS). Again, the sponge element (SE) has a smaller diameter than the rigid support (RS). The filter element (FE) may be affixed to the side wall at the periphery of the rigid support (RS).

Page 4

This page illustrates how things look when the sponge element (SE) and filter element (FE) are attached (or affixed) to the rigid support.

When the flange is flat, the sponge element (SE) may be compressed since the rigid support (RS), being rigid, does not conform/deform/bend as would the earlier-described plastic sheet (PS). This is especially true when filter diameter is small. Adding walls to rigid support (RS) (FIG. 9B) provides a recess having sufficient depth so that the sponge element (SE) will not be compressed. In other words, the recess (FIG. 9B) ought to be at least as deep as the sponge element (SE) is thick. This is because when the sponge element (SE) is very compressed the sponge pores will become compressed thereby increasing air flow resistance. The flange walls alleviate that by limiting, or avoiding, or reducing sponge compression.

In summary, Appendix-2 shows some embodiments of the filtration device (FD) which facilitate rigid mounting of the filtration device (FD) onto the facemask (M). In one embodiment, also shown in FIGS. 7, 7A, 7B, 8A, 8B, an external element (EE) associated with the filtration device (FD) screws into an internal element (IE) associated with the facemask (M). In another embodiment, also shown in FIG. 9, a rigid support (RS) is shown which may be considered to be an external element (EE) with an oversized flange (FL), said rigid support (RS) eliminating the need (substituting) for the plastic sheet (PS). Therefore, the flange (FL) of the rigid support (RS) should be the size (diameter) of the plastic sheet (PS) that it takes the place of (in lieu of).

Appendix-3

Included with this filing is a document (Appendix-3), which is incorporated by reference herein and forms part of the Specification (description) hereof. This document comprises 5 pages. This document contains text. Some changes or “editorial comments” may be presented herein. This document contains illustrations, which may be considered to be “Drawings” (or Figures of the Drawing), some of which correspond to drawing figures presented herein.

This document presents some additional or alternate embodiments of the invention. Some of these embodiments may reprise embodiments previously discussed, in their entirety or partially.

Page 1

This page illustrates a first embodiment (1.) showing rigid screw-on mounting of filter device to nebulizer mask.

On the top-left portion of this page are two images, an external mask view, of the filtration device (FD, or filter device) mounted to a nebulizer mask, with one filter on each side (left and right) of the mask.

To the right of that, on the top-right portion of the page is an internal mask view wherein a nut (N) can be seen. The nut (N) corresponds to the aforementioned internal element (IE) mentioned in FIGS. 7A, 7B, 8A. The nut may be one of the mating pair of internal and external elements IE and EE, respectively, allowing for rigid screw-on mounting of the filter device (FD) to the facemask (M, or FM).

The filtration device(s) illustrated here may be the filtration device described in Appendix-1 at page 2 thereof and/or in FIG. 9.

The bottom portion of the page shows the components of the filtration device (FD), as follows:
the filter media or filter element (FE)
the sponge, sponge element (SE)
a rigid support (RS)

Also shown is a nut (N) which is the internal element (IE) associated with the facemask (M)

Page 2

The image on the top-left portion of the page shows the assembled filter device (FD), two of which were shown on the previous page, from the filter or outlet side of the filtration device (FD).

The top-right portion of this page shows the internal element (IE) or nut (N) associated with the facemask (M) and the filtration device (FD) as viewed from the facemask or inlet side of the filtration device (FD).

There are three images on the bottom portion of the page which show the following.
The image on the left shows the filtration device (FD) as viewed from the inlet side of the filtration device. The threaded, tubular protrusion of the external element (EE) associated with the filtration device (FD) can be seen. The sponge element (SE) is visible.
The image in the middle shows the filtration device (FD) with the nut (N) associated with the facemask (F) mounted to the threaded, tubular protrusion of the external element (EE) associated with the filtration device (FD). The facemask (M) itself is omitted, for illustrative clarity.
The image on the right is a cross-sectional of the filtration device (FD) with nut (N) mounted (facemask omitted) which was shown in the previous image, through the section E-E.

Page 3

This page further illustrates an embodiment (2.) for mounting of the filter device (FD) to a non-rebreather facemask (M). This embodiment is a modification of the “stickie” embodiments such as shown in FIGS. 5A, 5B, 6A, 6B, wherein the filtration device (FD) is adhesively mounted to the facemask (M).

As shown in the left-hand image, a non-rebreather mask (M) may be fitted with valve flaps over the mask port(s) (P). The flaps allow air to be expelled by the user wearing the mask to pass through the port(s), while restricting air to be taken in through the port(s). For a non-rebreather mask, air (or oxygen) may be taken in via a separate inlet port of the mask. This is an external view of the mask (M), without the filtration device (FD) fitted thereto.

The mask (M) has two openings or ports, one port on each side (left and right) thereof. Each port is covered by a valve flaps, the flaps acting as check valves allowing air exhaled by a patient wearing the mask to be expelled (exhausted), without permitting air to be inhaled through the ports. In use, when the user is exhaling, the flaps need to move outward. In order to allow the flaps to freely move outward, the filtration device (FD) may be modified, as follows.

The image on the right side of the page (which is a cross-sectional view) shows that a cutout (CO) may be provided in the sponge element (SE) to allow the corresponding flap on the facemask (M) to deflect outward, unimpaired, when the patient is exhaling.

FIG. 10 illustrates this modification of a “stickie” embodiment of a filtration device (FD) wherein the sponge element (SE, SP) has a cutout or recess (CO) for allowing the flap of a non-rebreather mask to deflect outward when the patient is exhaling. This modification may be applied to the “stickie” embodiments of a filtration device shown in FIGS. 5A, 5B, 6A, 6B. In FIG. 10, the adhesive layer (AL) of FIGS. 5A, 5B and the flexible substrate (FS) and adhesive layer (AL) of FIGS. 6A, 6B are omitted, for illustrative clarity.

The cutout (CO) extends into the sponge element (SE) from the inlet side of the filtration device (FD), at least partially through the sponge element (SE) towards the outlet side of the filtration device (FD). The cutout (CO) need not extend across the entire width of the sponge element (SE). For a sponge element (SE) which is circular, the cutout (CO) may, for example, also be substantially circular, having a diameter which is a fraction of the overall diameter of the sponge element (SE).

In this embodiment, the plastic sheet (PS) is disposed on the inlet side of the filtration device (FD), with the sponge element (SE) disposed between the plastic sheet (PS) and the filter element (FE), in the manner shown in FIGS. 5A, 5B, 6A, 6B.

Page 4

This page further illustrates an embodiment for non-rebreather masks, such as shown on the previous page.

The two illustrations on the left side of the page show the sponge element (SE, SP) with a cutout to allow movement of the valve flap of the facemask (not shown). The left-hand illustration is a view of the filtration device (FD) from the inlet side (towards the mask). The right-hand illustration shows line A-A for the cross-sectional view of the filtration device (FD) shown on the illustration on the right side of the page. Here, it can be seen that the cutout (CO) to allow movement of the mask flap may extend only partially through the sponge element (SE) from the inlet side thereof. Of course, this cutout should be located appropriately over the flap so that the flap can move. The flap only needs to move slightly, to allow air exhaled by the patient to be expelled from the facemask.

Page 5

This page illustrates a third embodiment (3.) of filter with protective cover to prevent contamination due to contact with the filter element (FE) of the filtration device (FD). This is shown in the context of a filtration device employing a “stickie” interface, such as shown in FIGS. 5A, 5B, 6A, 6B. The protective cover can also be applied to the rigid support (RS) embodiment of FIG. 9 as well.

Generally, this embodiment is a modification of (or addition to) the original (“BG-131”) embodiment, such as shown in FIGS. 5A, 5B, 6A, 6B, wherein an additional sponge element (SE2) and protective cover (PC) are added to the outlet side of the filtration device (FD). All of the elements (PS, SE, FE, SE2, PPC) may be welded together, around their circumference. In this, and other embodiments disclosed herein, these elements are circular. It is within the scope of the invention that the elements are other than circular.

On the top portion of the page are two images.
The left-hand image is a view of the filtration device (FD) as viewed from the inlet (mask) side of the filtration device (FD).
The right-hand image is a view of the filtration device (FD) as viewed from the outlet side of the filtration device (FD).

In this embodiment, a protective cover (PC) is provided over the exposed outlet side of the filter element (FE), with the second sponge element (SE) disposed between the protective cover and the filter element (FE). The protective cover (PC) may simply be a second (additional) plastic sheet, similar to the first plastic sheet (PS), with perforations to allow air exhaled be the wearer of the mask to exit the filtration device (FD) on its outlet side. The protective cover may be a folded-over extension/portion of the first plastic sheet (PS).

On the bottom portion of the page are two images, both of which are cross-sectional views corresponding to the two images on the top portion of the page.
The left-hand image (labeled Original Embodiment) shows the original (“BG-131”) embodiment, with “stickie”, inlet side facing up, with the addition of the second sponge element (SE2) and protective cover (PC) on the outlet side of the filtration device (FD).
The right-hand image (labeled “Protective Cover”) shows the filtration device (FD), inverted, with its outlet side facing up. The protective cover (PC) has a number (such as 6) of perforations (PF) to allow air to exit the filtration device (FD). The second sponge element (SE2) can be seen disposed between the protective cover (PC) and the filter element (FE).

FIG. 11 shows the embodiment of the filtration device (FD) with a protective cover (PC) disposed on the outlet side of the filtration device (FD), with a second sponge element (SE2) disposed between the protective cover (PC) and the filter element (FE). A number (such as 6) of perforations (PF) are provided in the protective cover (PC). The protective cover (PC) and second sponge element (SE2) may be applied (added) to the “stickie” embodiments of a filtration device (FD) shown in FIGS. 5A, 5B, 6A, 6B. In FIG. 11, the adhesive layer (AL) of FIGS. 5A, 5B and the flexible substrate (FS) and adhesive layer (AL) of FIGS. 6A, 6B are omitted, for illustrative clarity. The cutout (CO) shown in FIG. 10 could be added to this embodiment. The protective cover (PC) and second sponge element (SE2) could be added to the rigid support (RS) embodiment of FIG. 9.

While the invention(s) may have been described with respect to a limited number of embodiments, these should not be construed as limitations on the scope of the invention(s), but rather as examples of some of the embodiments of the invention(s). Those skilled in the art may envision other possible variations, modifications, and implementations that are also within the scope of the invention(s), and claims, based on the disclosure(s) set forth herein.

What is claimed is:

1. A filtration device (FD) suitable to be mounted to a facemask (M) having a port (P), comprising:
a plastic sheet (PS) having an inlet port (IP);
a sponge element (SE) disposed behind the plastic sheet (PS);
a filter element (FE) disposed behind the sponge element (SE); and
an adhesive layer (AL) disposed on the plastic sheet (PS) about the inlet port (IP);
wherein the filtration device (FD) has an inlet side and an outlet side and can be mounted at its inlet side by the adhesive layer (AL) to the facemask with the inlet port (IP) of the filtration device (FD) aligned with the mask port (P).

2. The filtration device (FD) of claim 1, further comprising:
a flexible substrate (FS) disposed between the plastic sheet (PS) and the adhesive layer (AL) and having an opening aligned with the inlet port (IP) of the filtration device (FD).

3. The filtration device (FD) of claim 1, wherein:
the adhesive layer (AL) comprises a pressure-sensitive adhesive having a thickness of approximately 2 mil (0.002 in; 0.05 mm).

4. The filtration device (FD) of claim 1, wherein:
the plastic sheet (PS) comprises polyethylene.

5. The filtration device (FD) of claim 1, wherein:
the plastic sheet (PS) has a thickness of approximately 6 mil (0.006 inch; 0.15 mm).

6. The filtration device (FD) of claim 1, wherein:
the filter element (FE) comprises a pad type filter having a thickness of approximately 43 mil (0.043 inch; 1.1 mm).

7. The filtration device (FD) of claim 1, further comprising:
a protective cover (PC) disposed over the filter element (FE), on the outlet side of the filtration device (FD), to protect the filter element (FE) from contamination.

8. The filtration device (FD) of claim 7, further comprising:
perforations (PF) in the protective cover (PC) to allow air exhaled by a patient to exit the filtration device (FD).

9. The filtration device (FD) of claim 7, further comprising:
a second sponge element (SE2) disposed between the filter element (FE) and the protective cover (PC).

10. The filtration device (FD) of claim 7, wherein:
the protective cover (PC) comprises an additional plastic sheet or a folded-over extension of the (first) plastic sheet.

11. The filtration device (FD) of claim 1, further comprising:
a cutout (CO) extending into the sponge element (SE) from the inlet side of the filtration device (FD), at least partially through the sponge element (SE) towards the outlet side of the filtration device (FD) to allow a flap of a non-rebreather facemask to move outward when a patient wearing the facemask exhales. See FIG. 10.

12. The filtration device (FD) of claim 1, wherein:
in lieu of the adhesive layer (AL), the filtration device (FD) is provided with an external element (EE) capable of mating with an internal element (IE) associated with the facemask (M).

13. The filtration device (FD) of claim 12, wherein:
the external element (EE) is hollow tubular.

14. The filtration device (FD) of claim 12, wherein:
each of the internal element (IE) and external element (EE) comprises a hollow tubular portion and has a disc-like coaxial extension of its hollow tubular portion to permit secure mounting to an internal surface of the facemask (M) or filtration device (FD), respectively.

16. The filtration device (FD) of claim 12, wherein:
the external element (EE) has external threads and the internal element (IE) has internal threads so that the filtration device (FD) may be securely screwed onto the facemask (M).

17. The filtration device (FD) of claim 1, wherein:
in lieu of the adhesive layer (AL), the filtration device (FD) is provided with a rigid support (RS) in the form of a hollow tubular element having a flange (FL) which is the size of the plastic sheet (PS) that it is substituted for.

18. The filtration device (FD) of claim 17, wherein:
the hollow tubular portion of the rigid support (RS) has external threads for mating with internal threads of an internal element (IE) associated with the mask (M) so that the filtration device (FD) may be securely screwed onto the facemask (M).

19. The filtration device (FD) of claim 17, wherein:
the sponge element is smaller than the flange of the rigid support; and
the filter element is affixed to an outer annular region of the rigid support.

20. The filtration device (FD) of claim 17, wherein:
a recess is provided on the outlet side of the rigid support, forming a peripheral wall;
the sponge element is smaller than the flange of the rigid support;
the sponge element resides within the recess; and
the filter element is affixed to the wall of the rigid support.
